(19) United States
(12) Patent Application Publication
(10) Pub. No.: US 2025/0195508 A1
(43) Pub. Date: Jun. 19, 2025

(54) COMPOUNDS AND COMPOSITIONS FOR ANITMALARIAL THERAPEUTIC AND PROPHYLACTIC USE

(71) Applicants: UNIVERSITY OF CENTRAL FLORIDA RESEARCH FOUNDATION, INC., Orlando, FL (US); Dana-Farber Cancer Institute, Inc., Boston, MA (US); The Regents of the University of California, Oakland, CA (US); The Board of Trustees of the Leland Stanford Junior University, Stanford, CA (US)

(72) Inventors: Debopam CHAKRABARTI, Orlando, FL (US); Monica BOHMER, Orlando, FL (US); Nathanael GRAY, Stanford, CA (US); Lushun WANG, Stanford, CA (US); Jinhua WANG, Boston, MA (US); Jaeson Santos CALLA CHOQUE, Oakland, CA (US); Elizabeth WINZELER, Oakland, CA (US)

(21) Appl. No.: 18/843,885

(22) PCT Filed: Mar. 6, 2023
(86) PCT No.: PCT/US2023/014591

§ 371 Date: Sep. 4, 2024

Related U.S. Application Data
(60) Provisional application No. 63/316,835, filed on Mar. 4, 2022.

(51) Int. Cl. A61K 31/496; A61K 9/00; A61P 33/06; C07D 409/04

(52) U.S. Cl. CPC A61K 31/496; A61K 9/0019; A61P 33/06; C07D 409/04

(57) ABSTRACT

Malaria still afflicts about half of the world population causing more than 400,000 deaths, mostly children. The options for malaria therapy are increasingly becoming limited because of widespread drug resistance. Furthermore, drugs for prophylaxis are suboptimal. This disclosure reports the identification of type II protein kinase inhibitor compounds which have never been explored as antimalarial agents and which now have been discovered to possess therapeutic and prophylactic properties against malaria.

### CROSS-REFERENCE TO RELATED APPLICATIONS

This application is an international (PCT) application which claims the benefit of U.S. provisional application Ser. No. 63/316,835, filed 4 Mar. 2022. The entire contents of each of this prior application is hereby incorporated by reference as if fully set forth herein.

### GOVERNMENT FUNDING SUPPORT

This invention was made with government support under grant no. R01AI172022 awarded by the National Institutes of Health and grant no. AI152533 awarded by the National Institutes of Health. The government has certain rights in the invention.

### BACKGROUND OF THE INVENTION

### 1. Field of the Invention

This invention relates to the field of medicine and in particular to small molecule compounds for treatment and prophylaxis of malaria.

### 2. Background of the Invention

Malaria is a tropical, vector-borne protozoan disease, which is one of the most prevalent parasitic infections for mankind. Over 40% of the world's population is at risk for malaria. The malarial parasite is transmitted by mosquitoes in many tropical and subtropical regions. Human malaria is mainly caused by five species of protozoan parasites of the genus Plasmodium, with P. falciparum being the most virulent and fatal species. Malaria is initiated when Plasmodium sporozoites are transmitted to the human host during the blood feeding of infected female Anopheles mosquitos. Upon transmission, sporozoites invade hepatocytes, develop into merozoites, and eventually release into the bloodstream. The released merozoites replicate in the erythrocytes, causing malaria-associated clinical manifestations. Some merozoites differentiate into gametocytes. Transmission of the parasites to the vectors occurs when Anopheles mosquitos ingest the gametocytes during a blood meal, instigating the sexual sporogonic cycle.

The most common symptoms of malaria include a flu-like illness with fever, shivering, vomiting, nausea, joint pain, muscle aches, and headaches. The classical symptom of malaria is the cycle of sudden chill with shivering followed by fever and then sweating persisting for six to ten hours. Other symptoms experienced by malaria patients include dizziness, malaise, myalgia, abdominal pain, mild diarrhea, and dry cough. Other complications of severe malaria may occur and include splenomegaly, cerebral ischemia, hepatomegaly, hypoglycemia, hemoglobinuria, renal failure, pulmonary edema, and acidosis. Consequences of the infection include coma and death. The World Health Organization estimates that there were 241 million clinical episodes and 627,000 deaths from malaria in 2020, predominantly among children below age of five years and pregnant women in Africa.

### SUMMARY OF THE INVENTION

There is a great need in the art for compounds and methods for treatment and prophylaxis of malaria. In particular embodiments, the present invention relates to thiophene-pyridine compounds (type II kinase inhibitors) and derivatives, useful for malaria indications. Methods of use of these compounds also are disclosed.

In particular, embodiments of the invention include compounds of Formula I

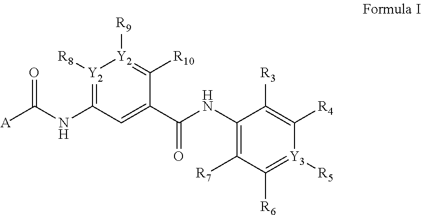

or a prodrug, stereoisomer, or pharmaceutically acceptable salt thereof,
wherein A is

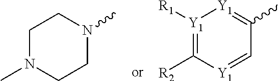

wherein each Y₁ is C or optionally one or two of Y₁ is N, wherein R¹ is no group, —H, —CH₃, —CN,

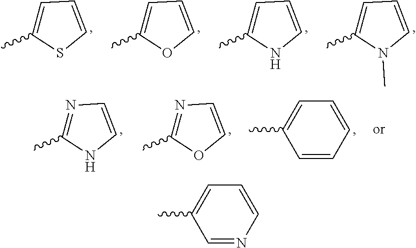

and wherein R₂ is no group, —H, or —NH₂;
wherein Y₂ is C or optionally one of Y₂ is N;
wherein Y₃ is C or N;
wherein R³ is —H;
wherein R⁴ is —H, —CF₃,

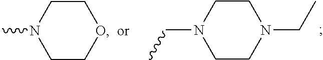

wherein R⁵ is no group, —H, or

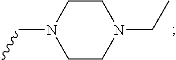

wherein R⁶ is —H, —CF₃,

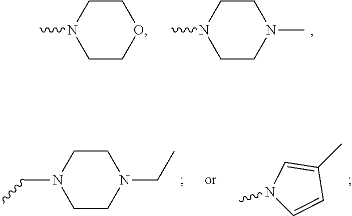

wherein R₇ is —H or —X;
wherein R₈ is no group, —H, —CH₃, or —X;
wherein R₉ is no group, —H, —CH₃, —C₂H₅, —CF₃, —CN, —OCH₃, or —X;
wherein R₁₀ is —H or —X; and
wherein X is-F, —Cl, or —Br.

The invention also includes as embodiments compounds according to Formula IIA or Formula IIB

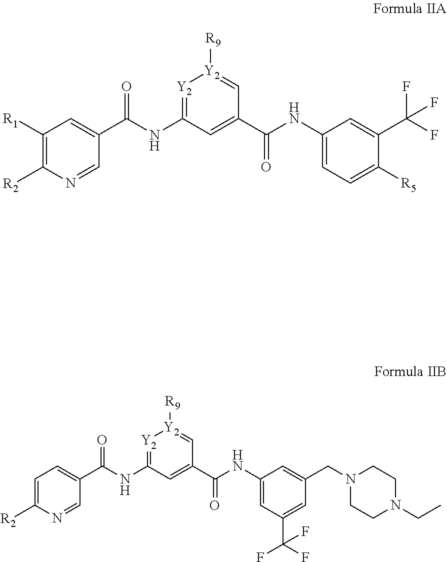

wherein R¹ is —H, —CN,

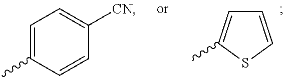

wherein R₂ is —H or —NH₂;
wherein Y₂ is C or optionally one of Y₂ is N;
wherein R⁵ is —H or

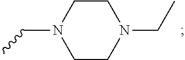

and
wherein R₉ is —H, —X, CH₃, or —OCH₃, or is absent when one of Y₂ is N.

Further embodiments of the invention include compounds according to Formula III

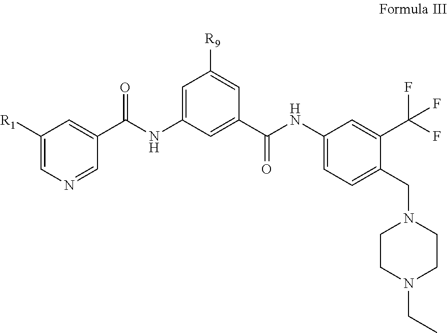

wherein R₉ is —H, —CH₃, —C₂H₅, —CF₃ or —X, wherein X is —Cl, —F, or —Br; and
wherein R¹ is —H, —CN,

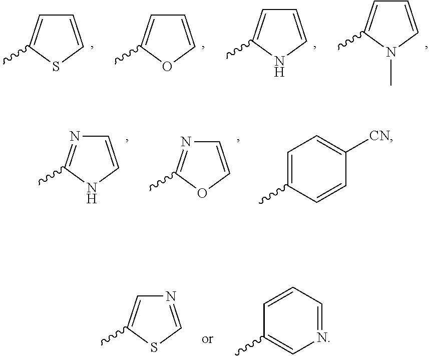

Further embodiments of the invention include compounds according to Formula IV

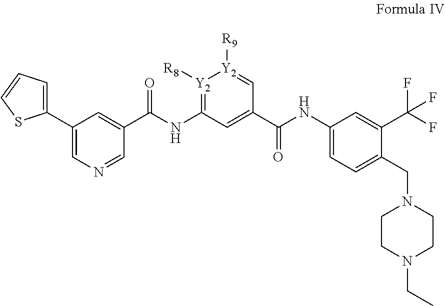

wherein Y₂ is C or optionally one of Y₂ is N;
wherein R₈ is —H or —CH₃; and
wherein R₉ is —CH₃, —C₂H₅, or —CN.

Further embodiments of the invention include compounds according to Formula V

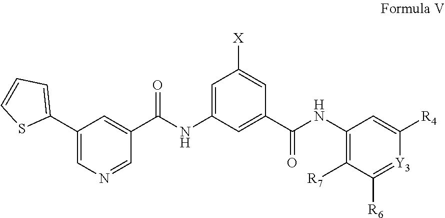

wherein Y₃ is C or N;
wherein R⁴ is-CF₃ or

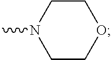

wherein R⁶ is —H,

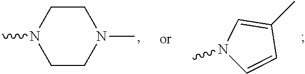

and
wherein R₇ is —H or —X, wherein X is —Cl, —F, or —Br.

Further embodiments of the invention include compounds according to Formula VI

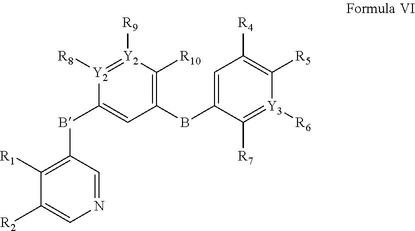

wherein R¹ is no group, —H, —CH₃, —CN,

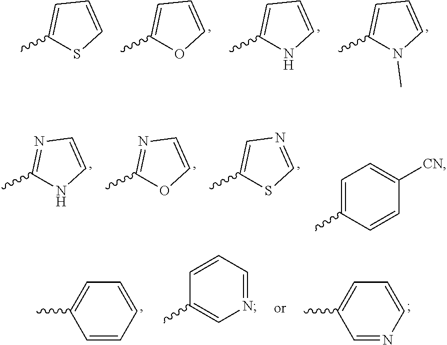

wherein R₂ is —H or —NH;
wherein B is —CO—NH—, —NH—CO—, or —NH—;
wherein B′ is —CO—NH—, —NH—CO—, or —NH—;
wherein Y₂ is C or optionally one of Y₂ is N;
wherein Y₃ is C or N;
wherein R⁴ is —H, —CF₃,

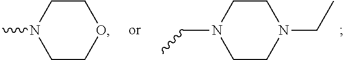

wherein R⁵ is —H, —CH₃, or

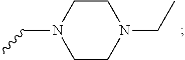

wherein R⁶ is —H, —CF₃,

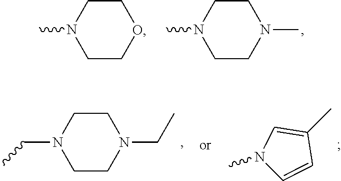

wherein R₇ is —H, —X;
wherein R₈ is no group, —H, —CH₃, or —X;
wherein R₉ is no group, —H, —CH₃, —C₂H₅, —CF₃, —CN, —OCH₃, or —X; and
wherein R₁₀ is —H or —X, wherein X is Cl, F, or Br.

Certain preferred compounds of the invention include those described herein where A is

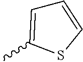

### and R₅ is

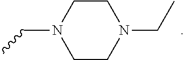

A preferred embodiment of the invention is

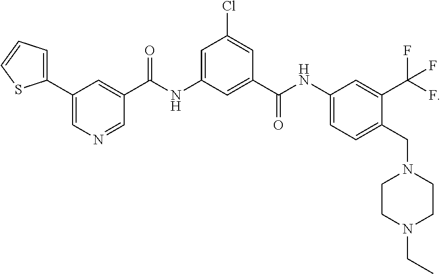 [SMILES: CCN1CCN(Cc2ccc(NC(=O)c3cc(Cl)cc(NC(=O)c4cncc(-c5cccs5)c4)c3)cc2C(F)(F)F)CC1]

Embodiments of the invention also included pharmaceutical compositions comprising a pharmaceutically acceptable carrier and one or more of the compounds described herein. Such compositions can be formulated for oral administration or for administration by injection.

Additional embodiments of the invention include methods for treatment or prevention of Plasmodium infection in a subject in need thereof, comprising administering an effective amount of one or more of the compounds or the compositions described herein to the subject in need. In preferred embodiments, the Plasmodium comprises P. berghei, P. falciparum, P. vivax, P. ovale, P. malariae, or P. knowlesi.

### BRIEF DESCRIPTION OF THE DRAWINGS

Certain embodiments are illustrated by way of example, and not by way of limitation, in the figures of the accompanying drawings.

FIG. 1 shows the structure of selected synthesized analogs of TL5-136.

FIG. 2 shows the structure of compounds according to embodiments of the invention with solvent exposure substitutions.

FIG. 3 shows the structure of compounds according to embodiments of the invention with substitutions in the middle ring.

FIG. 4 shows the structure of compounds according to embodiments of the invention including substitutions in the tail.

FIG. 5 shows the structure of compounds according to certain additional embodiments of the invention.

FIG. 6 presents data showing that TL5-135 eliminates malaria infection.

FIG. 7 presents data showing that TL5-135 exhibits prophylactic activity.

FIG. 8A presents data on the prophylactic and therapeutic activity of TL5-135 at a 50 mg/kg dose.

FIG. 8B presents data on the prophylactic and therapeutic activity of TK5-135 at a 15 mg/kg dose.

FIG. 9A presents survival data for mice infected with Pb-Luc malarial parasites.

FIG. 9B presents survival data for mice infected with Pb-Luc malarial parasites.

FIG. 10A, FIG. 10B, FIG. 10C, FIG. 10D, and FIG. 10E show the stage specific activity of and TL5-135. Control cell Giemsa staining is shown in FIG. 10A.

FIG. 11 shows the killing profiles of TL5-135, dihydroartemisinin (DHA) positive control, and atovaquone positive control as indicated.

### DETAILED DESCRIPTION OF EMBODIMENTS OF THE INVENTION

### 1. Overview

Thiophene-pyridine and related compounds have been discovered which are useful for the treatment and prophylaxis/prevention of malaria, based on the structure of TL5-135.

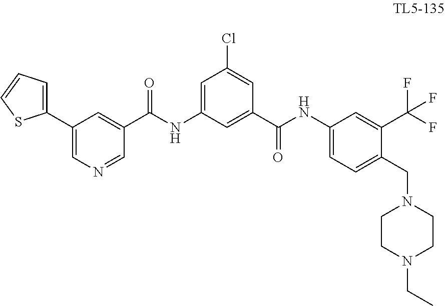 [SMILES: CCN1CCN(Cc2ccc(NC(=O)c3cc(Cl)cc(NC(=O)c4cncc(-c5cccs5)c4)c3)cc2C(F)(F)F)CC1]

### 2. Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods and materials are now described. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present disclosure are generally performed according to conventional methods well known in the art and as described in various general and more specific references that are cited and discussed through the present specification unless otherwise indicated. All publications mentioned herein are incorporated herein by reference.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

As used herein, the term “about” generally means plus or minus 1%, 2%, 3%, 4%, 5%, 6%, 7%, 8%, 9%, or 10% of the recited value, so that, for example, “about 0.125” means 0.125±0.0125, and “about 1.0” means 1.0±0.1, using 10%. Notwithstanding that the numerical ranges and parameters setting forth the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

As used herein, the terms “administering” or “administration” of an agent, compound, drug, or peptide to a subject refers to any route of introducing or delivering to a subject a compound to perform its intended function. The administering or administration can be carried out by any suitable route as deemed appropriate by the practitioner, including orally, intranasally, parenterally (intravenously, intramuscularly, intraperitoneally, or subcutaneously), rectally, or topically. Administering or administration includes self-administration and the administration by another.

As used herein, the terms “composition” or “pharmaceutical composition” comprise one or more of the compounds described herein as active ingredient(s), or a pharmaceutically acceptable salt(s) thereof, and preferably also contain a pharmaceutically acceptable carrier and optionally also contain other therapeutic ingredients. The compositions include compositions suitable for oral, rectal, ophthalmic, pulmonary, nasal, dermal, topical, parenteral (including subcutaneous, intramuscular and intravenous) or inhalation administration, or any method of administration deemed appropriate by the practitioner for a particular subject. The most suitable route in any particular case will depend on the nature and severity of the conditions being treated and the nature of the active ingredient(s). The compositions may be presented in unit dosage form and prepared by any of the methods well-known in the art of pharmacy. Dosage regimes may be adjusted for the purpose to improving the therapeutic response. For example, several divided dosages may be administered daily or the dose may be proportionally reduced over time. A person skilled in the art normally is able to determine the effective dosage amount and the appropriate regime.

As used herein, the term “analog” refers to a compound having a structure similar to that of another compound but differing from the other compound with respect to a certain component or substituent. The compound may differ in one or more atoms, functional groups, or substructures, which may be replaced with other atoms, groups, or substructures. In one aspect, such structures possess at least the same or a similar therapeutic efficacy.

As used herein, “derivative” refers to a compound derived or obtained from another and containing essential elements of the parent compound. In one aspect, such a derivative possesses at least the same or similar therapeutic efficacy as the parent compound.

As used herein, the term “pharmaceutically acceptable salt” is intended to include nontoxic base addition salts. Suitable salts include those derived from organic and inorganic acids such as, without limitation, hydrochloric acid, hydrobromic acid, phosphoric acid, sulfuric acid, methanesulfonic acid, acetic acid, tartaric acid, lactic acid, sulfinic acid, citric acid, maleic acid, fumaric acid, sorbic acid, aconitic acid, salicylic acid, phthalic acid, and the like. The term “pharmaceutically acceptable salt” as used herein is also intended to include salts of acidic groups, such as a carboxylate, with such counterions as ammonium, alkali metal salts, particularly sodium or potassium, alkaline earth metal salts, particularly calcium or magnesium, and salts with suitable organic bases such as lower alkylamines (methylamine, ethylamine, cyclohexylamine, and the like) or with substituted lower alkylamines (e.g. hydroxyl-substituted alkylamines such as diethanolamine, triethanolamine or tris(hydroxymethyl)-aminomethane), or with bases such as piperidine or morpholine.

As used herein, the term “prodrug” refers to a compound that is converted to a therapeutically active compound after administration, and the term should be interpreted as broadly herein as is generally understood in the art. Generally, but not necessarily, a prodrug is inactive or less active than the therapeutically active compound to which it is converted. For example, a methyl ester prodrug can be converted to a free carboxylic acid drug in vivo via the action of non-specific serum esterases.

As used herein, As used herein, the term “stereoisomer” refers to a compound which has the identical chemical constitution but differs with regard to the arrangement of the atoms or groups in space.

As used herein, As used herein, the terms “treating” or “treatment” or “alleviation” refers to both therapeutic treatment and prophylactic or preventative measures, wherein the objective is to prevent or slow down (lessen) the targeted pathologic condition or disorder. The term “treatment regimen” refers to a course of treatment or a series of doses administered to a subject over time. Thus, treatment includes administration of a compound to cure the disease, to reduce the length of infection, to reduce the severity of the disease, to inhibit the disease or a symptom thereof, to reduce the parasitemia in a subject suffering from the disease, and the like.

As used herein, the term “preventing” means causing the clinical symptoms of the disease state not to worsen or develop, e.g., inhibiting the onset of disease, in a subject that may be exposed to or predisposed to the disease state, but does not yet experience or display symptoms of the full disease state, e.g., malaria. Thus, prevention and/or prophylaxis includes administration of a compound to completely prevent the occurrence of the disease, to reduce the occurrence of the disease in a population, to reduce the likelihood of occurrence of the disease in a subject, to reduce the severity of the disease when it does occur, to inhibit the disease or a symptom thereof, to reduce parasitemia in a subject, and the like.

According to certain embodiments, provided are methods of preventing or treating malaria in a subject or preventing or treating a subject exhibiting a symptom of malaria by administering effective amounts of one or more compounds described herein. Malaria in humans typically produces a string of recurrent attacks, or paroxysms, each of which has three stages—chills, followed by fever, and then sweating. Along with chills, the person is likely to have headache, malaise, fatigue, muscular pains, occasional nausea, vomiting, and diarrhea. Within an hour or two, the body temperature rises, and the skin feels hot and dry. Then, as the body temperature falls, a drenching sweat begins. The person, feeling tired and weak, is likely to fall asleep. Thus, a subject exhibiting one, two or more of the foregoing symptoms is considered a subject in need.

As the term is used herein, the term “subject” includes any animal, preferably a mammal, including mammals such as a human, a laboratory animal, a companion animal, and a livestock animal. Preferably, the subject is human, however the invention contemplates treatment of animals such as rats, mice and rabbits, livestock such as cattle, sheep, pigs and the like, and companion animals such as dogs, cats and other pets. A “subject in need” refers to any mammal, particularly a human, who suffers from malaria or a symptom of malaria, has been diagnosed with malaria, or is likely to become infected by the malarial parasite, for example by being in an area endemic to the disease.

As used herein, The terms “pharmaceutically acceptable carrier, excipient, vehicle, or diluent” refer to a medium which does not interfere with the effectiveness or activity of an active ingredient and which is not toxic to the hosts to which it is administered. A carrier, excipient, vehicle, or diluent includes but is not limited to binders, adhesives, lubricants, disintegrants, bulking agents, buffers, and miscellaneous materials such as absorbents, taste-masking agents, pH modifiers, and the like, that may be needed in order to prepare a particular composition.

As used herein, “anti-malarial” or “anti-malarial activity” includes any activity that decreases the infectivity, the reproduction, or inhibits the progress of the lifecycle of a malaria parasite. “Anti-malarial activity” also includes inhibition of the growth of malaria infection by all of the means observed with current anti-malarial drugs.

As used herein, the term “anti-malarial agent” refers to any compound according to the various embodiments, compounds referred to in the Tables below and the accompanying figures, and any combinations, prodrugs, pharmaceutically acceptable salts, analogs, and derivatives thereof. The compositions and methods described herein are useful for the treatment and/or prevention of malaria. The determination of a therapeutically effective dose is well within the capability of those skilled in the art.

As used herein, a “therapeutically effective dose” refers to that amount of active ingredient which causes reduction or elimination of malaria in a subject and includes a single dose as well as a course of doses or a regimen of doses administered to a subject.

As used herein, the term “effective amount,” “amount effective,” “therapeutically effective amount,” or the like, means an amount or number of effective dosages administered for periods of time necessary to achieve the desired result. For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans, as is understood by persons of skill in the art.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀. These data are commonly used by the person of ordinary skill to determine an appropriate dose or range of doses.

The half maximal inhibitory concentration (IC₅₀) is a measure of the potency of a substance in inhibiting a specific biological or biochemical function. IC₅₀ is a quantitative measure that indicates how much of a particular inhibitory substance is needed to inhibit, in vitro, a given biological process or biological component by 50%.

As used herein, The half maximal effective concentration (EC₅₀) is a statistical estimate of the concentration of a toxicant in the ambient medium necessary to produce a particular effect in 50% of a very large population under specified conditions.

### 3. Embodiments of the Invention

### A. Chemical Compounds and Pharmacological Data Therefor.

Several kinase inhibitor compounds were here discovered to possess antimalarial activity. One compound, TL5-135, was shown to eliminate established Plasmodium. berghei infections in mice when administered orally and prevent infection when administered intravenously. TL-135 was found to block ring to trophozoite maturation and exhibited a killing effect. These are behaviors that meet many of the Medicine for Malaria Venture's Target Product Profiles for antimalarial compounds.

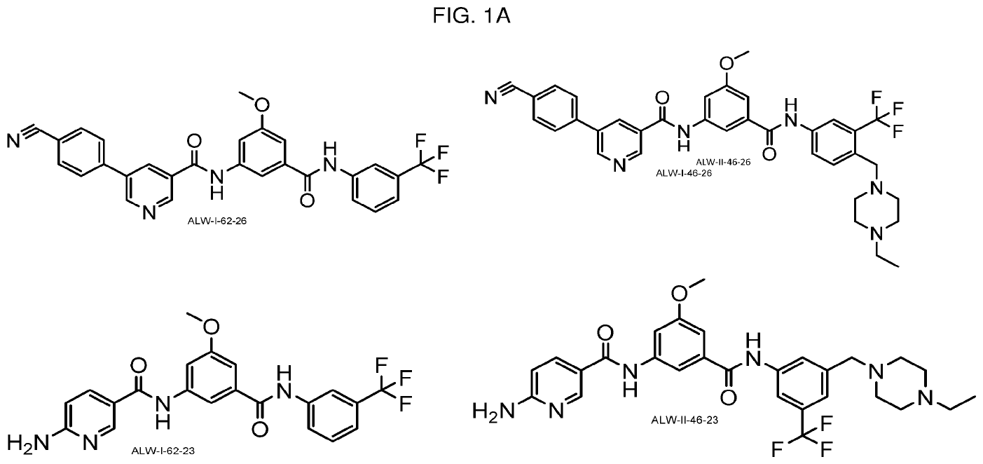

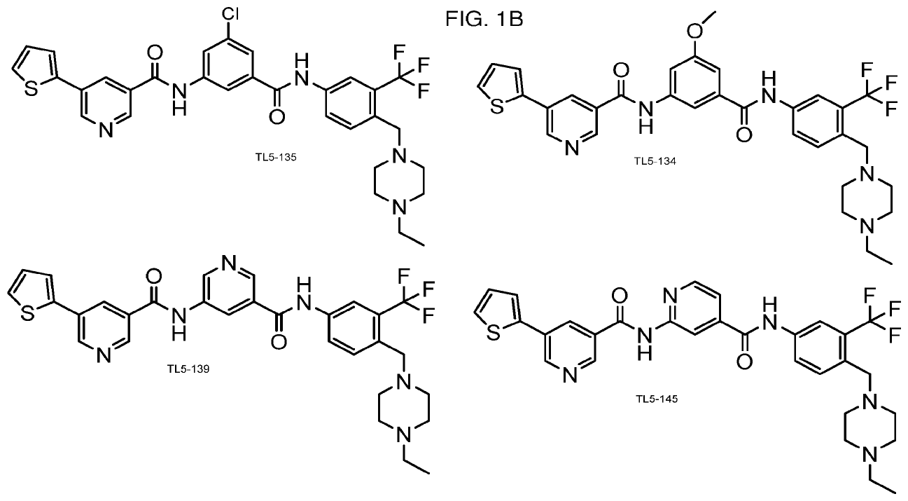

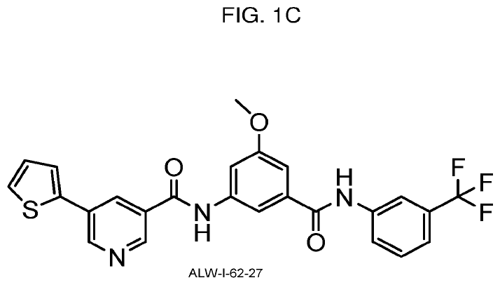

Several analogs were synthesized based on the structure of TL5-135 for further study. The synthesized analogs shown in FIG. 1 and Table 1, below were found to have an EC₅₀<500 nM in a CQ^{R} Dd2 strain and an IC₅₀>10,000 nM in Human EPHA2. For the data in Table 1, compounds were subjected to in vitro biochemical kinase assays done using recombinant human ephrin type-A receptor 2 (EPHA2) (expressed in insect cells) in a Z′-LYTE® FRET-based kinase assay. P. falciparum Dd2 EC₅₀ was determined using the standard 72 hour SYBR Green I assay (Dd2 Proliferation Assay) as known in the art. Accordingly, the disclosed novel compounds possess antiplasmodial therapeutic and prophylactic properties. The structures of compoundsTL5-134, TL5-139, and TL5-145 are shown below.

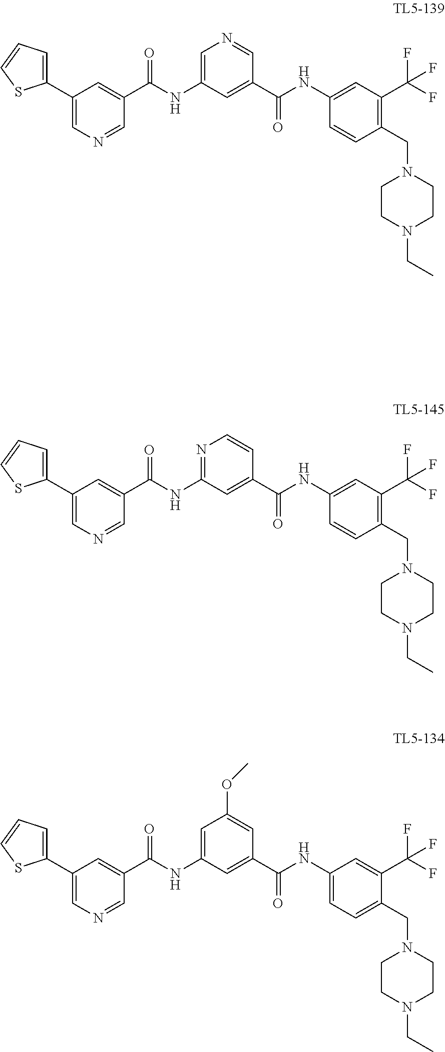 [SMILES: CCN1CCN(Cc2ccc(NC(=O)c3cncc(NC(=O)c4cncc(-c5cccs5)c4)c3)cc2C(F)(F)F)CC1 ; CCN1CCN(Cc2ccc(NC(=O)c3ccnc(NC(=O)c4cncc(-c5cccs5)c4)c3)cc2C(F)(F)F)CC1 ; CCN1CCN(Cc2ccc(NC(=O)c3cc(NC(=O)c4cncc(-c5cccs5)c4)cc(OC)c3)cc2C(F)(F)F)CC1]

TABLE 1
Pharmacologic Data for TL5-135 and Example Analogs.

| | | Human | Antiplasmodial |
|---|---|---|---|
| Molecule | | EPHA2 IC₅₀ | Dd2 EC₅₀ |
| Name | SMILES | (nM) ± SD | (nM) ± SD |
| TL5-135 | CCN1CCN(CC2═CC═C(NC(═O)C3═CC(NC(═O) | >10000 | 80 |
| | C4═CC(═CN═C4)C4═CC═CS4)═CC(Cl)═C3) | | |
| | C═C2C(F)(F)F)CC1 | | |
| ALW-II-46-26 | CCN1CCN(CC2═CC═C(NC(═O)C3═CC(NC(═O) | Not | 130 |
| | C4═CC(═CN═C4)C4═CC═C(C═C4) | determined | |
| | C#N)═CC(OC)═C3)C═C2C(F)(F)F)CC1 | | |
| TL5-139 | CCN1CCN(CC2═CC═C(NC(═O)C3═CC(NC(═O) | >10000 | 191 |
| | C4═CC(═CN═C4)C4═CC═CS4)═CN═C3) | | |
| | C═C2C(F)(F)F)CC1 | | |
| TL5-134 | CCN1CCN(Cc2ccc(NC(═O)c3cc(NC(═O) | >10000 | 341 |
| | c4cncc(c4)-c4cccs4)cc(OC)c3)cc2C(F)(F)F)CC1 | | |
| TL5-145 | CCN1CCN(CC2═CC═C(NC(═O)C3═CC(NC(═O) | >10000 | 391 |
| | C4═CC(═CN═C4)C4═CC═CS4)═NC═C3) | | |
| | C═C2C(F)(F)F)CC1 | | |
| ALW-II-46-23 | CCN1CCN(CC2═CC(═CC(NC(═O)C3═CC(NC(═O) | ND | 495 |
| | C4═CC═C(N)N═C4)═CC(OC)═C3)═C2)C(F) | | |
| | (F)F)CC1 | | |
| ALW-I-62-27 | COC1═CC(NC(═O)C2═CN═CC(═C2) | ND | ND |
| | C2═CC═CS2)═CC(═C1)C(═O) | | |
| | NC1═CC═CC(═C1)C(F)(F)F | | |
| ALW-I-62-26 | COC1═CC(═CC(NC(═O)C2═CC(═CN═C2) | ND | ND |
| | C2═CC═C(C═C2)C#N)═C1)C(═O) | | |
| | NC1═CC═CC(═C1)C(F)(F)F | | |
| ALW-I-62-23 | COC1═CC(═CC(NC(═O)C2═CC═C(N) | ND | ND |
| | N═C2)═C1)C(═O)NC1═CC═CC(═C1)C(F)(F)F | | |

ND = not determined.

According to the invention, compounds of Formula I are embodiments:

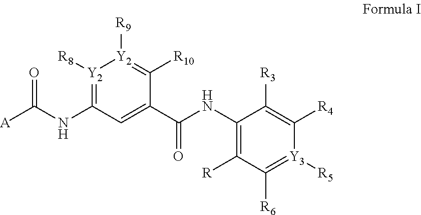

wherein A is

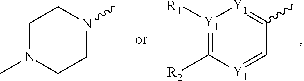

wherein each Y₁ is C or optionally one or two of Y₁ is N, and wherein R¹ is no group, —H, —CH₃, —CN,

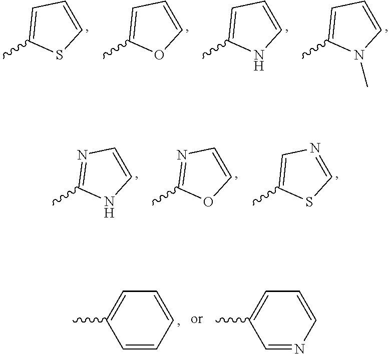

and wherein R₂ is —H or —NH₂;
wherein Y₂ is C or optionally one of Y₂ is N;
wherein Y₃ is C or N;
wherein R³ is —H;
wherein R⁴ is —H, —CF₃,

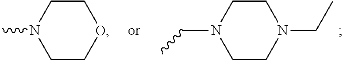

wherein R⁵ is no group, —H, or

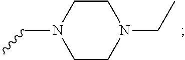

wherein R⁶ is —H, —CF₃,

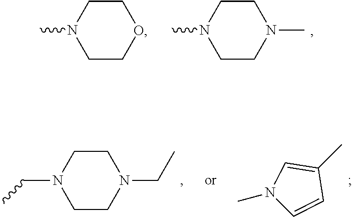

wherein R₇ is —H or —X;
wherein R₈ is no group, —H, —CH₃, or —X;
wherein R₉ is no group, —H, —CH₃, —C₂H₅, —CF₃, —CN, —OCH₃, or —X;
wherein R₁₀ is —H or —X; and
wherein X is-F, —Cl, or —Br.

Analogs, derivatives, prodrugs, stereoisomers, or pharmaceutically acceptable salts of the compounds according to Formula I are also within the scope of the present disclosure and are contemplated for use in or with the invention. In certain embodiments, compounds can be selected from the formulas shown in FIGS. 1-5.

### In a Specific Preferred Embodiment the Compound is

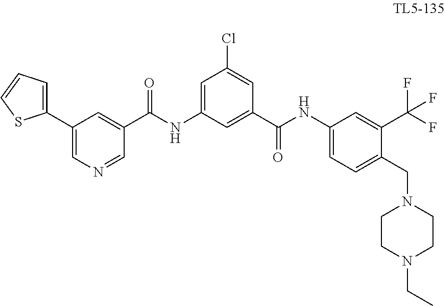 [SMILES: CCN1CCN(Cc2ccc(NC(=O)c3cc(Cl)cc(NC(=O)c4cncc(-c5cccs5)c4)c3)cc2C(F)(F)F)CC1]

or an analog, derivative, prodrug, stereoisomer, or pharmaceutically acceptable salt thereof.

Various embodiments of the invention include compounds, and salts, stereoisomers, prodrugs, analogs and derivatives thereof, according to Formula IIA or IIB. See FIG. 1 for examples. Analogs, derivatives, prodrugs, stereoisomers, or pharmaceutically acceptable salts of the compounds according to Formula IIA and Formula IIB are also within the scope of the present invention.

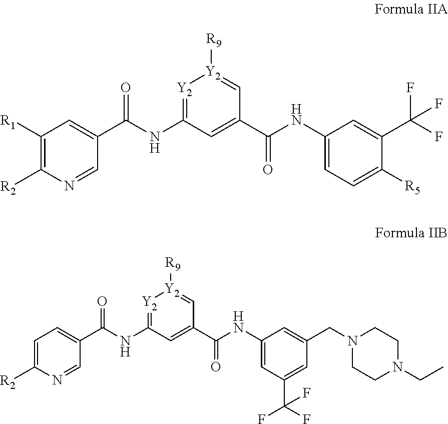

wherein R¹ is —H, —CN,

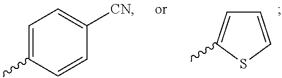

wherein R₂ is —H or —NH₂;
wherein Y₂ is C or optionally one of Y₂ is N;
wherein R⁵ is —H or

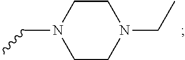

and
wherein R₉ is —H, —X, CH₃, or —OCH₃, or is absent when one of Y₂ is N.

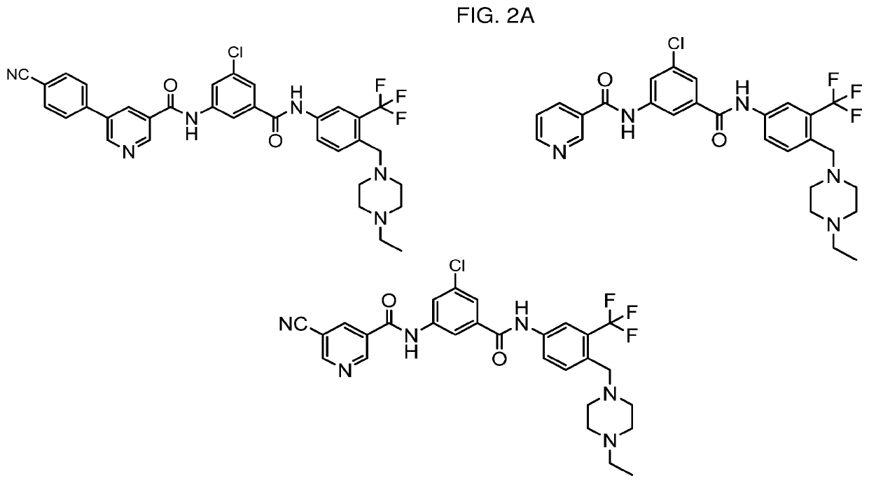

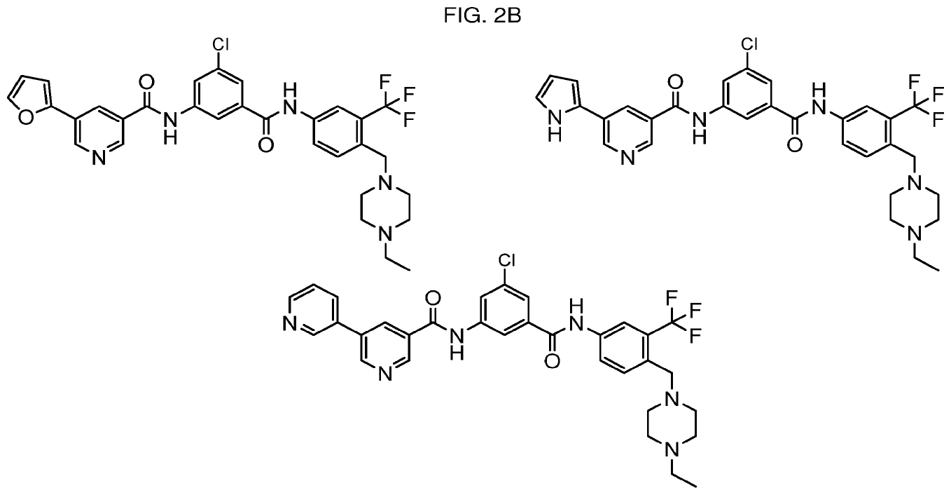

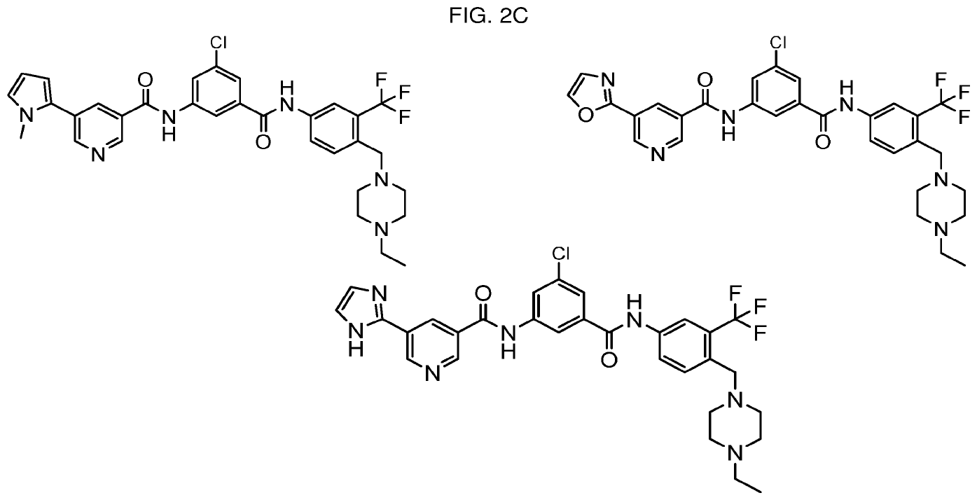

Various embodiments of the invention include compounds, and salts, stereoisomers, prodrugs, analogs and derivatives thereof, according to Formula III. See FIG. 2 for examples. Analogs, derivatives, prodrugs, stereoisomers, or pharmaceutically acceptable salts of the compounds according to Formula III are also within the scope of the present invention.

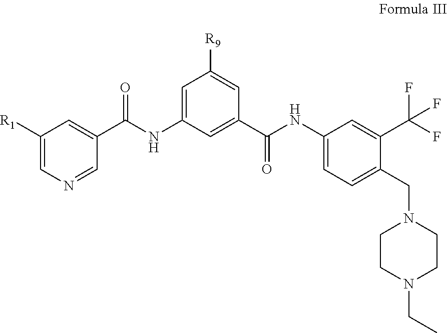

wherein R₉ is —H, —CH₃, —C₂H₅, —CF₃, or —X, wherein X is —Cl, —F, or —Br; and
wherein R¹ is —H, —CN,

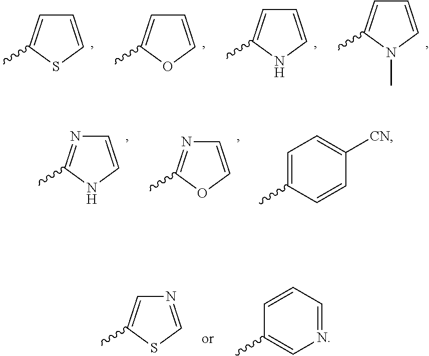

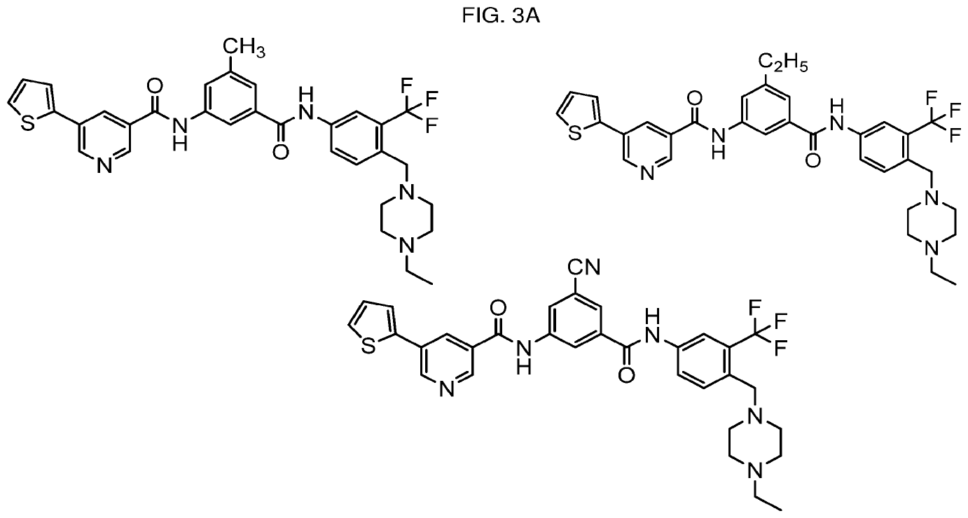

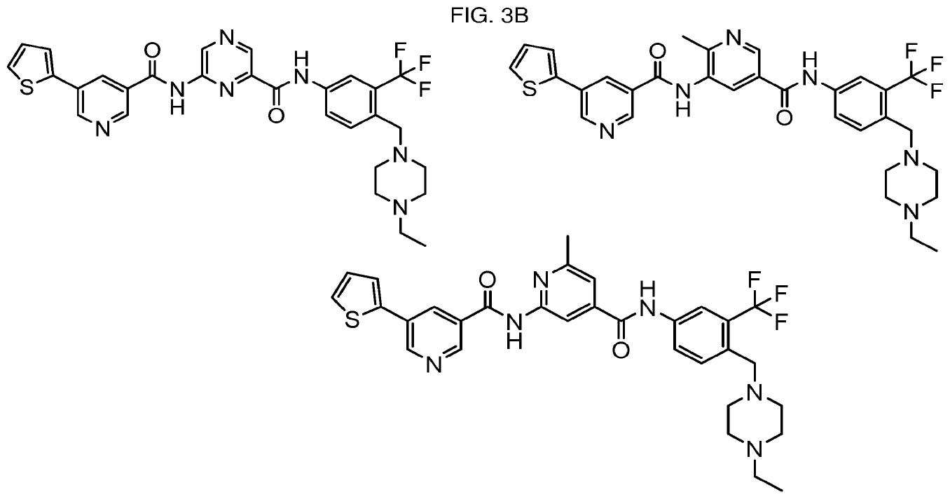

Various embodiments of the invention include compounds, and salts, stereoisomers, prodrugs, analogs and derivatives thereof, according to Formula IV. See FIG. 3 for examples. Analogs, derivatives, prodrugs, stereoisomers, or pharmaceutically acceptable salts of the compounds according to Formula IV are also within the scope of the present invention.

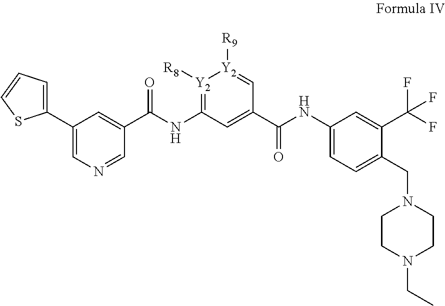

wherein Y₂ is C or optionally one of Y₂ is N;
wherein R₈ is —H or —CH₃; and
wherein R₉ is CH₃, —C₂H₅, or —CN.

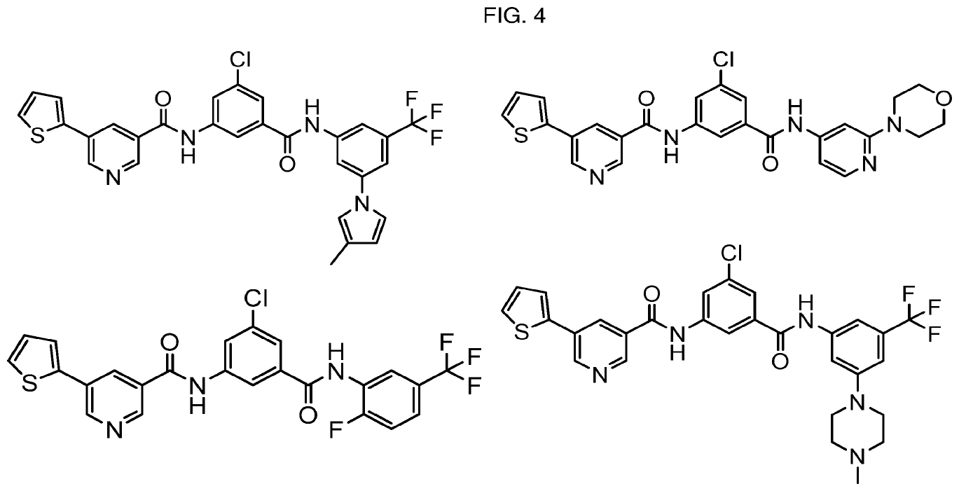

Various embodiments of the invention include compounds, and salts, stereoisomers, prodrugs, analogs, derivatives, and pharmaceutically acceptable salts thereof, according to Formula V. See FIG. 4 for example compounds. Analogs, derivatives, prodrugs, stereoisomers, or pharmaceutically acceptable salts of the compounds according to Formula V are also within the scope of the present invention.

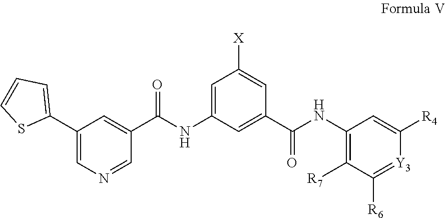

wherein Y₃ is C or N;
wherein R⁴ is-CF₃ or

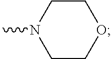

wherein R⁶ is —H,

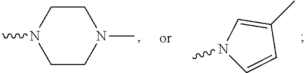

and
wherein R₇ is —H or —X, wherein X is —Cl, —F, or —Br.

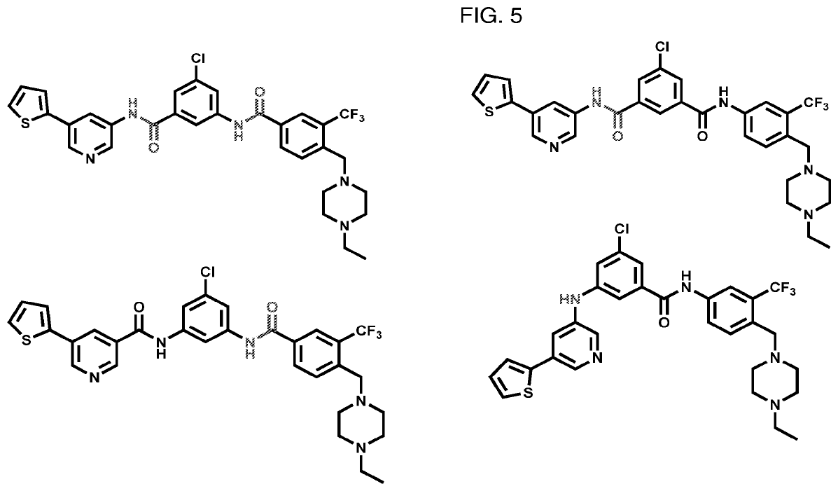

Various embodiments of the invention include compounds, and salts, stereoisomers, prodrugs, analogs and derivatives thereof, according to Formula VI. See FIG. 5. Analogs, derivatives, prodrugs, stereoisomers, or pharmaceutically acceptable salts of the compounds according to Formula IV are also within the scope of the present invention.

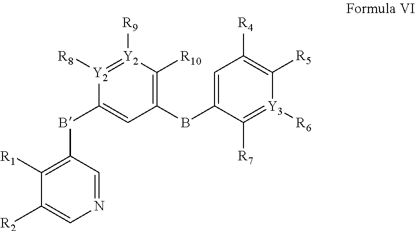

wherein R¹ is no group, —H, —CH₃, —CN,

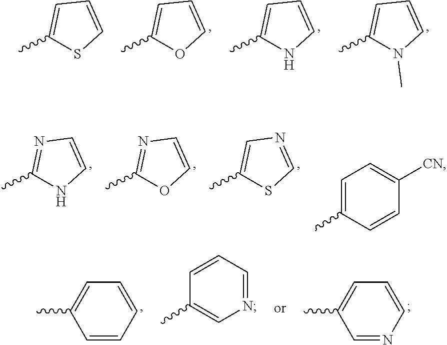

wherein R₂ is —H or —NH;
wherein B is —CO—NH—, —NH—CO—, or —NH—;
wherein B′ is —CO—NH—, —NH—CO—, or —NH—;
wherein Y₂ is C or optionally one of Y₂ is N;
wherein Y₃ is C or N;
wherein R⁴ is —H, —CF₃,

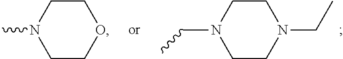

wherein R⁵ is —H, —CH₃, or

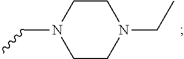

wherein R⁶ is —H, —CF₃,

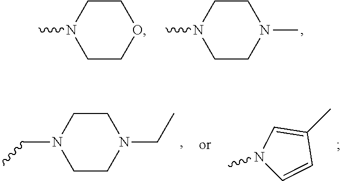

wherein R₇ is —H, —X;
wherein R₈ is no group, —H, —CH₃, or —X;
wherein R₉ is no group, —H, —CH₃, —C₂H₅, —CF₃, —CN, —OCH₃, or —X;
wherein R₁₀ is —H or —X, wherein X is Cl, F, or Br.

Various embodiments of the invention include compounds, and salts, stereoisomers, prodrugs, analogs and derivatives thereof of the compounds shown below. See Table 2 for pharmacological data.

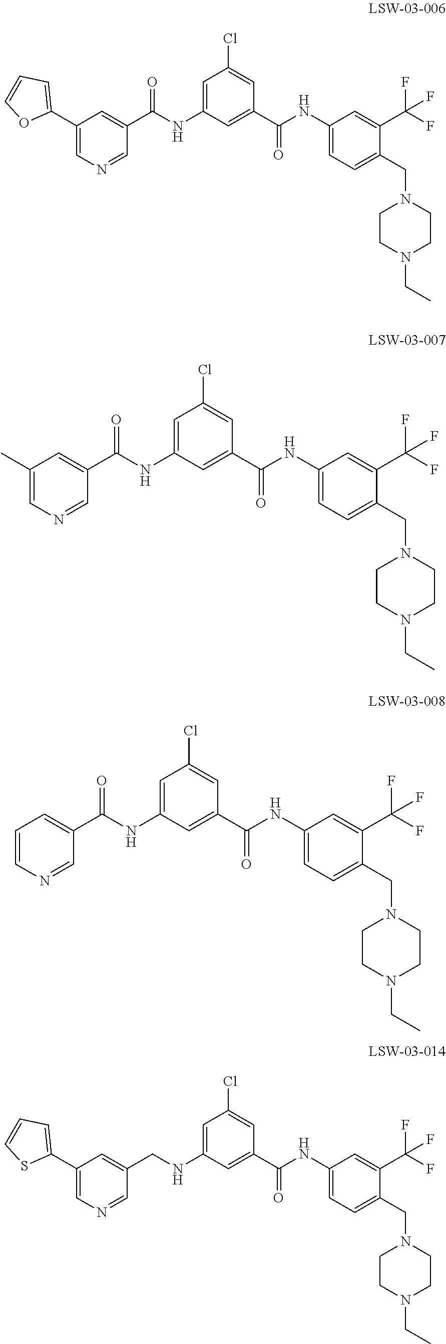

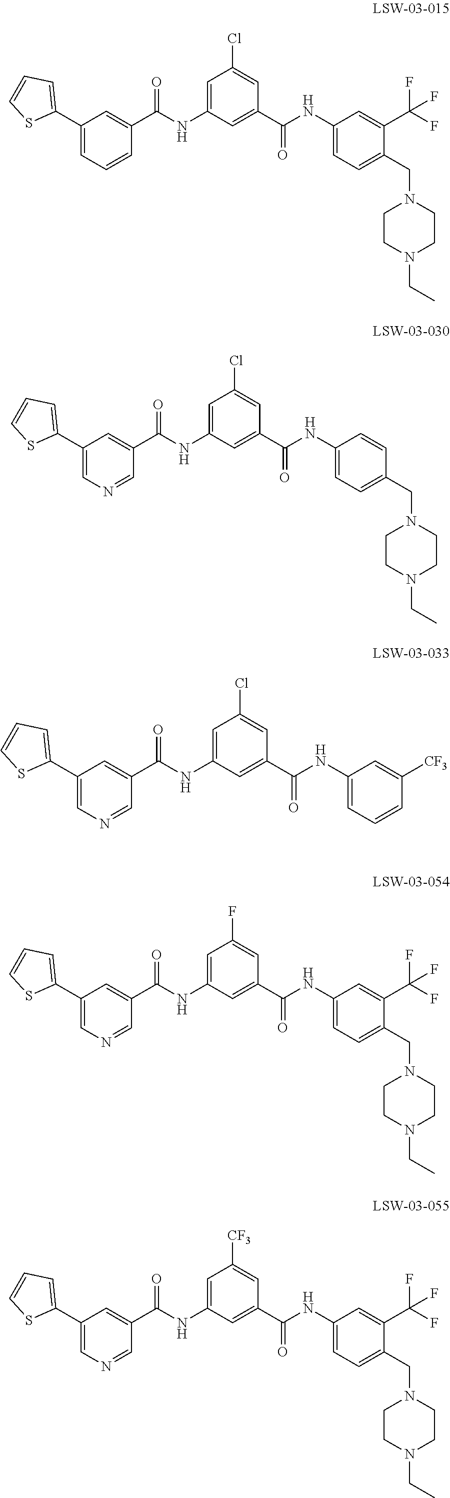 [SMILES: CCN1CCN(Cc2ccc(NC(=O)c3cc(Cl)cc(NC(=O)c4cccc(-c5cccs5)c4)c3)cc2C(F)(F)F)CC1 ; CCN1CCN(Cc2ccc(NC(=O)c3cc(Cl)cc(NC(=O)c4cncc(-c5cccs5)c4)c3)cc2)CC1 ; O=C(Nc1cccc(C(F)(F)F)c1)c1cc(Cl)cc(NC(=O)c2cncc(-c3cccs3)c2)c1 ; CCN1CCN(Cc2ccc(NC(=O)c3cc(F)cc(NC(=O)c4cncc(-c5cccs5)c4)c3)cc2C(F)(F)F)CC1 ; CCN1CCN(Cc2ccc(NC(=O)c3cc(NC(=O)c4cncc(-c5cccs5)c4)cc(C(F)(F)F)c3)cc2C(F)(F)F)CC1]

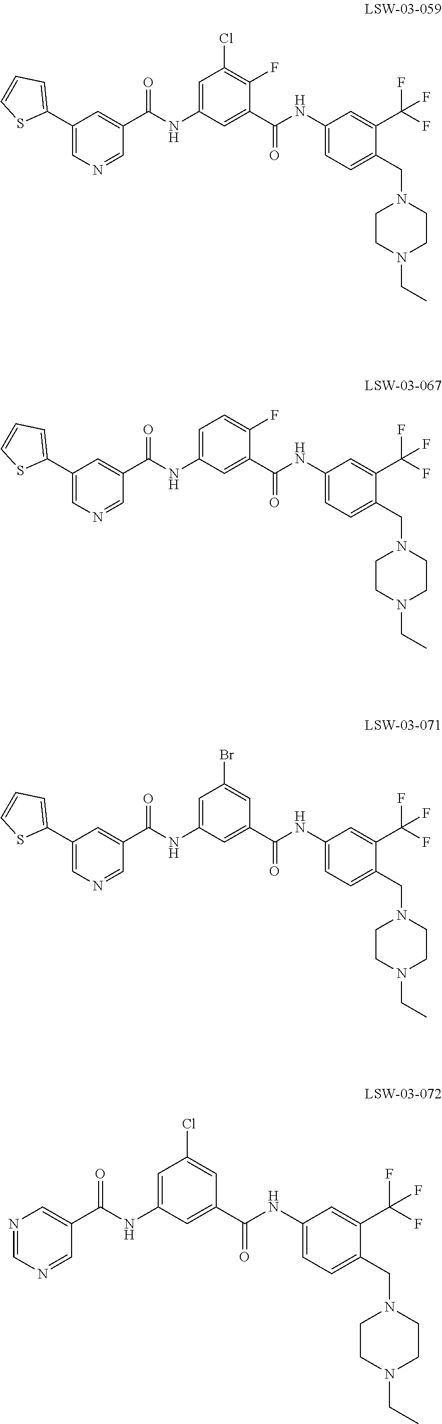 [SMILES: CCN1CCN(Cc2ccc(NC(=O)c3cc(NC(=O)c4cncc(-c5cccs5)c4)cc(Cl)c3F)cc2C(F)(F)F)CC1 ; CCN1CCN(Cc2ccc(NC(=O)c3cc(NC(=O)c4cncc(-c5cccs5)c4)ccc3F)cc2C(F)(F)F)CC1 ; CCN1CCN(Cc2ccc(NC(=O)c3cc(Br)cc(NC(=O)c4cncc(-c5cccs5)c4)c3)cc2C(F)(F)F)CC1 ; CCN1CCN(Cc2ccc(NC(=O)c3cc(Cl)cc(NC(=O)c4cncnc4)c3)cc2C(F)(F)F)CC1]

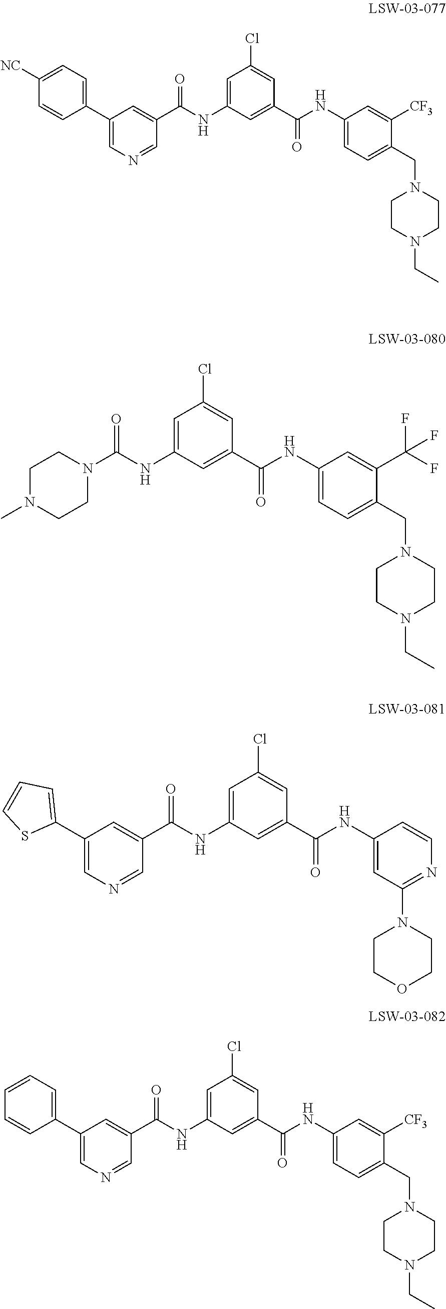

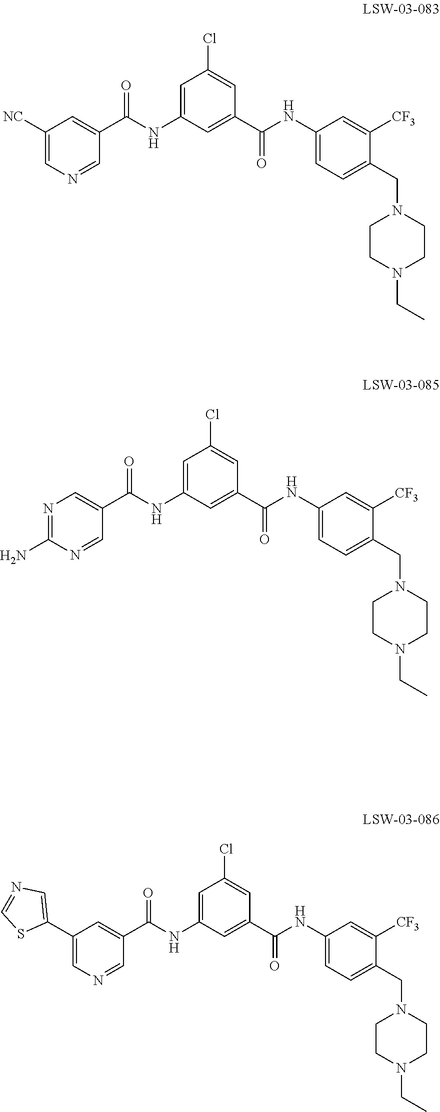 [SMILES: CCN1CCN(Cc2ccc(NC(=O)c3cc(Cl)cc(NC(=O)c4cncc(C#N)c4)c3)cc2C(F)(F)F)CC1 ; CCN1CCN(Cc2ccc(NC(=O)c3cc(Cl)cc(NC(=O)c4cnc(N)nc4)c3)cc2C(F)(F)F)CC1 ; CCN1CCN(Cc2ccc(NC(=O)c3cc(Cl)cc(NC(=O)c4cncc(-c5cncs5)c4)c3)cc2C(F)(F)F)CC1]

TABLE 2
EC50 Data for Compounds according to the Invention.

| Compound | Dd2 EC₅₀ (nM) ± SD |
|---|---|
| LSW-03-006 | 97.2 ± 23.3 |
| LSW-03-007 | 164 ± 14.7 |
| LSW-03-008 | 407 ± 56.0 |
| LSW-03-014 | 315 ± 18.7 |
| LSW-03-015 | 314 ± 17.4 |
| LSW-03-030 | 99.6 ± 8.6 |
| LSW-03-033 | 422 ± 36.0 |
| LSW-03-054 | 99.5 ± 12.7 |
| LSW-03-055 | 86.6 ± 2.92 |
| LSW-03-059 | 104 ± 8.67 |
| LSW-03-067 | 867 ± 68.3 |
| LSW-03-071 | 108 ± 9.08 |
| LSW-03-072 | 330 ± 27.4 |
| LSW-03-077 | 43.8 ± 7.9 |
| LSW-03-080 | 2.12E+3 ± 286 |
| LSW-03-081 | 732 ± 124 |
| LSW-03-082 | ND |
| LSW-03-083 | ND |
| LSW-03-085 | ND |
| LSW-03-086 | ND |

### B. Pharmaceutical Formulations and Administration

Various embodiments of the invention relate to pharmaceutical compositions comprising an effective amount of one or more of the compounds disclosed here or to various analogs of those compounds or combinations, derivatives, prodrugs, stereoisomers, or pharmaceutically acceptable salts thereof. The pharmaceutical compositions exhibit antiplasmodium potency against P. berghei, P. falciparum, P. vivax, P. ovale, P. malariae, or P. knowlesi. According to various embodiments, the pharmaceutical compositions may further comprise a pharmaceutically acceptable carrier and/or a conjunctive anti-malarial agent.

The present disclosure specifically includes pharmaceutical compositions and formulations of the compounds described here which contain an active agent as described herein and a pharmaceutically acceptable carrier or excipient. In typical embodiments, pharmaceutical compositions comprise therapeutically effective amounts of the inventive compounds, which amounts treat or prevent Plasmodium infection in a subject as a single dose or as part of a series of doses or a treatment regimen. Pharmaceutical compositions for use in the present methods include therapeutically effective amounts of one or more of the inventive compounds, in an amount sufficient to prevent or treat the diseases described herein in a subject, formulated for systemic administration. In an optional embodiment, one or more of the inventive compounds may be co-administered with at least one other active agent.

Thus, pharmaceutical compositions comprising one or more compounds as described herein also include one or pharmaceutically acceptable carriers. Such compositions may contain any excipient known in the art of pharmaceutics to function in making the composition suitable for administration to a subject. For example, excipients can function as a filler, a carrier, a binder, a disintegrant, a glidant, a preservative, a stabilizing agent, a wetting agent or surfactant, a pH modifier, an emulsifier, a solvent, an antimicrobial, an osmotic agent, and the like as known in the pharmaceutical arts, or may serve several of such functions.

The formulations can be prepared for any suitable route of administration, including liquid, solid, or gas formulations. Formulations such as solutions, suspensions, lipid emulsions, and the like are suitable for injection into the subject, including intravenous, intraarterial, intramuscular, subcutaneous, intraperitoneal, and the like, in a bolus dose or an infusion. Solid formulations can be prepared for administration orally, or as a suppository, implant, transdermal patch, a powder inhalant, and the like. Preferred administration is by intravenous or intraperitoneal injection.

The pharmaceutical compositions of this disclosure may be in a variety of forms, which may be selected according to the preferred modes of administration. These include, for example, solid, semi-solid and liquid dosage forms such as tablets, lozenge, pills, powders, liquid solutions or suspensions, suppositories, and injectable and infusible solutions. The preferred form depends on the intended mode of administration and therapeutic application.

For oral administration, compounds according to embodiments of the invention can be formulated as dispersible tablet, orally disintegrating tablet, effervescent tablet, chewable tablet, sprinkle granules, oral solution, dry suspension or dry syrup for reconstitution, quick melt wafers, lozenge, or chewing gum. Examples of suitable excipients for solid dosage forms for oral administration include, water, saline, dextrose, glycerol, pharmaceutical grades of mannitol, lactose, starch, magnesium stearate, sodium saccharin, cellulose, magnesium carbonate, and the like. These compositions typically contain 1%-95% of active ingredient, preferably 2%-70% active ingredient.

Aqueous solution compositions can be packaged for use as is or for dilution before use, filtered under aseptic conditions and lyophilized to be reconstituted with a sterile aqueous solution, such as water or saline solution, prior to administration. The compositions can contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc. Additionally, lipidic suspension compositions can include lipid-protective agents which protect lipids against free-radical and lipid-peroxidative damages on storage. Lipophilic free-radical quenchers, such as α-tocopherol and water-soluble iron-specific chelators, such as ferrioxamine, are suitable for inclusion as well. The pharmaceutical preparations optionally are sterilized by conventional, well known sterilization techniques.

Formulations for parenteral administration by injection include aqueous and non-aqueous sterile injection solutions, which optionally contain anti-oxidants, buffers, bacteriostats, and components that render the formulation isotonic with the blood of the intended recipient; and aqueous and non-aqueous sterile suspensions for injection which optionally include suspending agents and thickening agents. The formulations can be presented in unit-dose or multi-dose containers, for example prepared syringes or injector pens, sealed ampoules and vials, and may be stored in a freeze-dried (lyophilized) condition requiring only the addition of the sterile liquid carrier, for example saline or water-for-injection, immediately prior to use. Extemporaneous injection solutions and suspensions may be prepared from sterile powders, granules and tablets of the kind previously described.

The disclosure has references to specific embodiments. It will, however, be evident that various modifications and changes may be made thereto without departing from the broader spirit and scope of the disclosure. The specification and drawings are, accordingly, to be regarded in an illustrative rather than a restrictive sense. The disclosure is illustrated herein by the experiments described above and by the following examples, which should not be construed as limiting. The contents of all references, pending patent applications and published patents, cited throughout this application are hereby expressly incorporated by reference.

### C. Methods of Use

Methods of using the compounds described herein are contemplated as part of this invention. The compounds, or pharmaceutical compositions containing the compounds, are administered to a subject. For the prevention or treatment of disease, the appropriate dosage will depend on the severity of the disease, whether the drug is administered for protective or therapeutic purposes, previous therapy, the patient's clinical history and response to the drugs and the discretion of the attending physician. The formulations also can contain more than one of the inventive active compounds and/or contain additional active agents useful in the treatment or prevention of malaria. Such additional agents suitably are present in combination in amounts that are effective for the purpose intended. It is understood that the methods and uses of the embodiments of the invention disclosed here can be employed for prophylaxis/prevention as well as (more suitably) in the treatment of subjects suffering from Plasmodium infection. Thus, the inventive compounds can be administered with additional compounds (1) simultaneously in a single combination dosage form, (2) simultaneously in separate dosage forms, or (3) sequentially.

The duration of treatment with the compounds of embodiments of the invention can extend over any suitable period of time as determined by the clinician, depending on the condition of the subject, with the treatment preferably continuing until the Plasmodium infection is sufficiently reduced or eliminated. The progress of therapy is easily monitored by conventional techniques and assays that may be used to adjust dosage and the dosage regimen to achieve a desired therapeutic effect.

The subject is preferably a human but can be non-human as well. A suitable subject for treatment can be any individual who is suspected of having, has been diagnosed as having, or is at risk of developing a Plasmodium infection. A suitable subject for prophylaxis can be any individual who resides in a region endemic for malaria or is visiting such an area.

These and other features, aspects, and advantages of various embodiments will become better understood with reference to the following description, figures, examples and claims.

### 4. EXAMPLES

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1. General Methods and Assays

### A. TL5-135 and Synthesized Analog Testing.

In vitro biochemical kinase assays were done using recombinant human EPHA2 (expressed in insect cells) in a Z′-LYTE® FRET-based kinase assay. P. falciparum Dd2 EC₅₀ was determined using the standard 72 h SYBR Green I assay.

### B. Rate of Killing Assay

Asynchronous P. falciparum Dd2 cultures were plated at 1% parasitemia, 4% hematocrit into a 24-well plate, incubated at 37° C. in 5% CO₂. Wells were treated with 10×EC₅₀ of compound for 12, 24, or 48 hours before the compound was removed. Every 24 hours thereafter for 4 days total, culture samples were washed in 0.5% BSA and stained with 1×SYBR Green I and 60 μM MitoTracker Deep Red FM for 20 minutes at 37° C. After another wash in 0.5% BSA, samples were resuspended in PBS and analyzed via flow cytometry with a CytoFLEX S™, with 100,000 events recorded per sample. For gating, the following controls were used: uninfected RBCs, untreated culture, DHA-treated culture, and untreated culture without MitoTracker stain. Viable parasite percentage was determined by finding the fraction of SYBR Green/MitoTracker double positive cells from the total events. Analysis was performed with FlowJo software Version 10.

### C. SYBR Green Proliferation Assay (Dd2 Proliferation Assay).

Asynchronous blood stage P. falciparum Dd2 cultures were plated at 1% parasitemia and 1% hematocrit in a 96-well plate and incubated with serial dilutions of compound for 72 hours at 37° C. in 5% CO₂. DMSO concentrations were kept at a sub-lethal percentage (<0.2%). Five micromolar chloroquine was used as a negative parasite growth control. Plates were then frozen at −80° C. and thawed, and 100 μL SYBR Green I-containing lysis buffer (20 mM Tris-HCl, 5 mM EDTA, 0.8% Triton X-100, 0.08% saponin, and 0.0001% SYBR Green I) was added to the wells. After incubation for 45 minutes at room temperature protected from light, fluorescence emission was read with a multimode microplate reader, at 485 nm excitation and 530 nm emission. Results were normalized using untreated and chloroquine-treated controls, and EC₅₀ was determined with CDD Vault.

### D. Stage Specificity Assay

Asexual stage P. falciparum cultures were synchronized by MACS magnetic column, followed 3 hours later by treatment with 5% sorbitol to select for rings. Six hours after invasion, parasites were plated onto a 96-well plate at 1% parasitemia and 1% hematocrit, maintained at 37° C. in 5% CO₂, and compound was added at 5×EC₅₀ to the 6 HPI wells. Every 12 hours, until reinvaded rings (54 HPI), thin blood smear slides for Giemsa-stain were taken, and samples were washed and fixed in 4% paraformaldehyde in PBS with 0.04% glutaraldehyde. At the conclusion of the timepoints, all samples were permeabilized with 0.25% Triton X-100 before being treated with 50 μg/mL RNase A and stained with 500 nM YOYO-1. Samples were then washed, and flow cytometric analysis was performed with a CytoFLEX S™, collecting 100,000 events per sample. Analysis was performed with FlowJo™ software Version 10.

### E. Antimalarial Assays

TL5-135 was dissolved or resuspended in a suspending vehicle consisting of 30% DMSO, 1% methylcellulose, 0.5% TWEEN-80, and 68.5% ultrapure water. GNF179 was resuspended in 30% DMSO, 30% PEG400, 5% TWEEN-80, 35% saline, and administered intravenously.

For prophylaxis assays, a single dose of 15 mg/mg body weight was administrated 6 hours before infection. For therapeutic assays, in a 4-day's Peters test, 4 doses (each 15 mg/kg) were administrated 6, 18, 30, and 42 hours post infection.

The antimalarial activity was measured as a percent reduction in parasitemia from day one until day twelve post-infection. All mice treated with TL5-135 and GNF179 as prophylactic treatment were still healthy and alive after 22 days.

Mice treated with GNF179 as a therapeutic also were healthy and without parasites. However, the mice treated with TL5-135 as a therapeutic had about 2.4% parasitemia after 12 days post-infection (all negative control mice died between days 11-13). The parasitemia on day 21 (21 days post-infection) for the group of mice was 35%.

### Example 2. Chemical Synthesis of Inventive Compounds

### A. General Synthetic Pathway (Used to Synthesize Compounds LSW-03-030, 033, 054, 055, 059, 067, 071, 081, 082, 083, and 086)

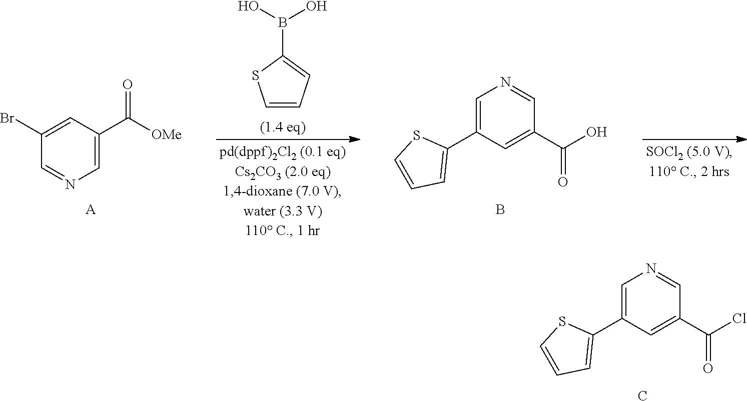

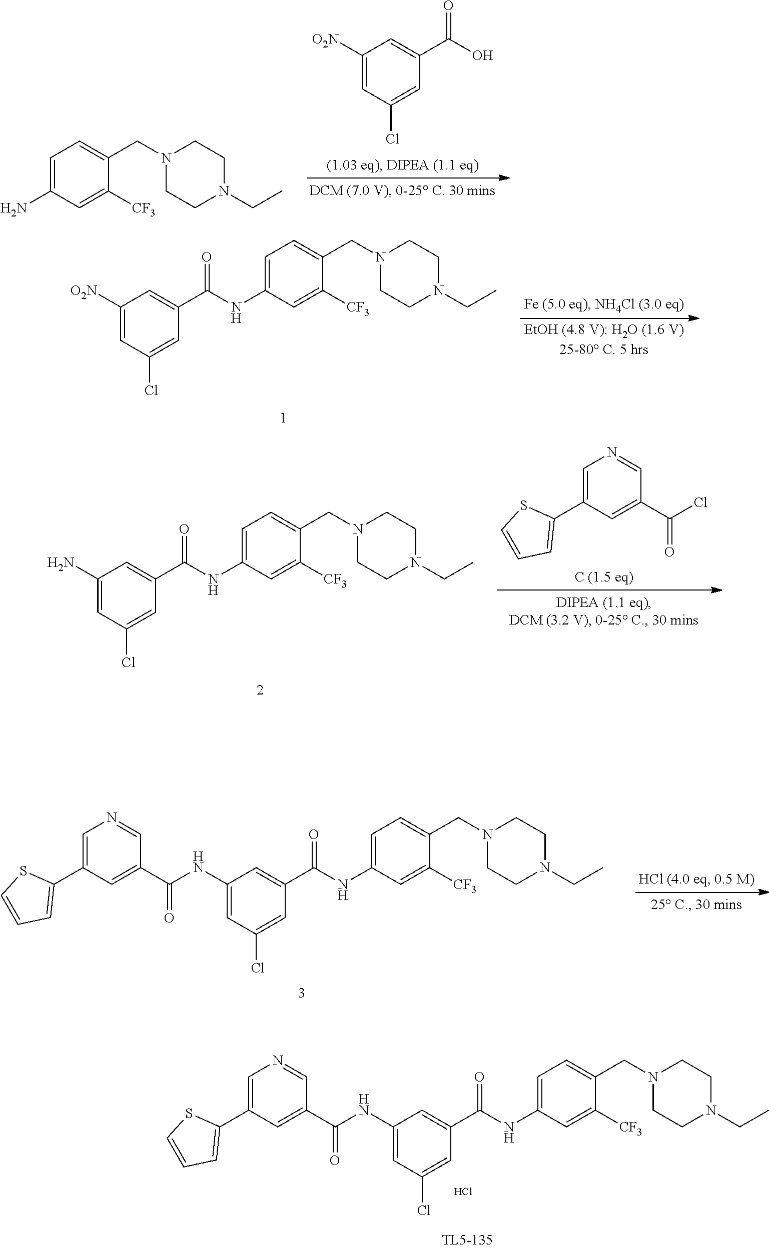

### B. Synthesis of Compound B

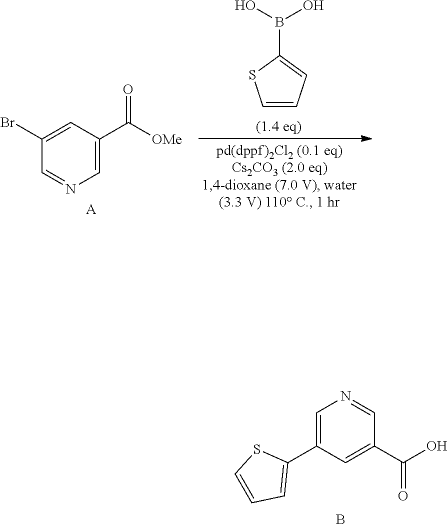

To a solution of A (9.00 g, 41.6 mmol, 1.0 eq) in 1,4-dioxane (60 mL) and H₂O (30 mL) thiophen-2-ylboronic acid (7.46 g, 58.3 mmol, 1.4 eq), Pd(dppf)Cl₂ (3.05 g, 4.17 mmol, 0.1 eq) and Cs₂CO₃ (27.15 g, 83.3 mmol, 2.0 eq) was added. The mixture was stirred at 110° C. for 1 hour. LC-MS showed compound A was consumed completely and one main peak with the desired MS was detected. The reaction mixture was concentrated under reduced pressure to remove 1,4-dioxane. The residue was diluted with H₂O (30 mL) and extracted with EtOAc (50 mL, 30 mL). The organic layers were washed with water (30 mL). The combined aqueous layers were diluted with hydrochloride acid (pH=2-3). The precipitated solid was filtered and dried under reduced pressure to give B (6.0 g, 29.24 mmol, 70.1% yield) as a white solid. ¹H NMR (400 MHZ, DMSO-d₆): δ (ppm) 9.13 (d, J=2.0 Hz, 1H), 8.97 (d, J=1.6 Hz, 1H), 8.39 (s, 1H), 7.75 (dd, J=23.2, 3.2 Hz, 2H), 7.22 (t, J=4.8 Hz, 1H).

### C. Synthesis of Compound C

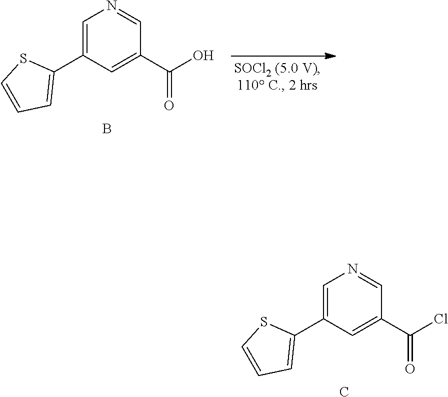

Compound B (3 g, 14.6 mmol, 1.0 eq) was dissolved in SOCl₂ (15 mL) at 25° C. The mixture was stirred at 110° C. for 2 hours. The reaction mixture was concentrated under reduced pressure to remove SOCl₂ (15 mL) to give compound C (3 g, 13.4 mmol, 91.7% yield) as a yellow solid.

### D. Synthesis of Compound 1

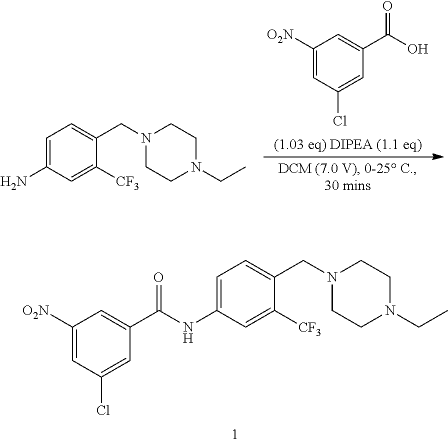 [SMILES: O=C(O)c1cc(Cl)cc([N+](=O)[O-])c1 ; CCN1CCN(Cc2ccc(N)cc2C(F)(F)F)CC1 ; CCN1CCN(Cc2ccc(NC(=O)c3cc(Cl)cc([N+](=O)[O-])c3)cc2C(F)(F)F)CC1]

To a solution of 4-((4-ethylpiperazin-1-yl)methyl)-3-(trifluoromethyl) aniline (4.00 g, 13.9 mmol, 1.0 eq) in DCM (28 mL), compound 3-chloro-5-nitrobenzoic acid (3.15 g, 14.3 mmol, 1.03 eq) and N,N-Diisopropylethylamine (DIPEA) (1.98 g, 15.3 mmol, 2.67 mL, 1.1 eq) were added at 0° C. The mixture was stirred at 25° C. for 0.5 hour. LCMS showed that 4-((4-ethylpiperazin-1-yl)methyl)-3-(trifluoromethyl) aniline was consumed completely and one main peak with desired MS was detected. The reaction was poured to water (15 mL) and the reaction was extracted with DCM (2×50 mL). The organic phase was dried over Na₂SO₄ and filtered under reduced pressure to give Compound 1 (4.2 g, 8.92 mmol, 64.07% yield) as a white solid. ¹H NMR (400 MHZ, MeOD): δ (ppm) 8.77 (s, 1H), 8.49 (s, 1H), 8.40 (s, 1H), 8.16 (s, 1H), 8.02 (d, J=8.4 Hz, 1H), 7.80 (d, J=8.4 Hz, 1H), 3.78 (s, 2H), 2.25-3.24 (m, 10H) 1.35 (t, J=7.6 Hz, 3H).

### E. Synthesis of Compound 2

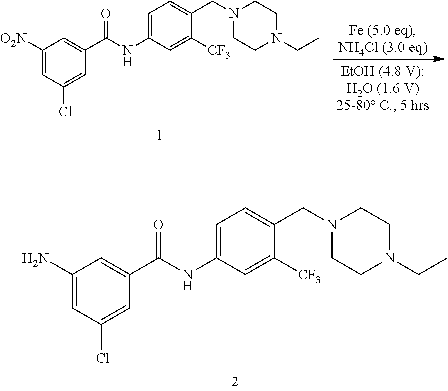 [SMILES: CCN1CCN(Cc2ccc(NC(=O)c3cc(Cl)cc([N+](=O)[O-])c3)cc2C(F)(F)F)CC1 ; CCN1CCN(Cc2ccc(NC(=O)c3cc(N)cc(Cl)c3)cc2C(F)(F)F)CC1]

To a solution of Compound 1 (2.50 g, 5.31 mmol, 1.0 eq) in EtOH (12 mL) and H₂O (4.0 mL), NH₄Cl (852 mg, 15.9 mmol, 3.0 eq) and Fc (1.48 g, 26.6 mmol, 5.0 eq) was added at 25° C. The mixture was stirred at 80° C. for 5 hours. TLC (DCM:MeOH=0:1, Product R_{f}=0.12) indicated that Compound 1 was consumed completely one new spot formed. The reaction was clean according to TLC. The reaction mixture was filtered and concentrated under reduced pressure to remove MeOH. The reaction was poured into water (15 mL) and extracted with ethyl acetate (50 mL, 30 mL). The combined organic phases were washed with saturated brine (10 mL), dried over Na₂SO₄, filtered and concentrated in vacuum to give a Compound 2 (2 g, 4.54 mmol, 85.44% yield) as a white solid. ¹H NMR (400 MHZ, DMSO): δ (ppm) 10.45 (s, 1H), 8.18 (d, J=2.0 Hz, 1H), 8.06 (d, J=8.4 Hz, 1H), 7.70 (d, J=8.4 Hz, 1H), 7.04-7.10 (m, 2H), 6.79 (t, J=2.0 Hz, 1H), 5.72 (s, 2H), 3.65 (s, 2H), 2.75-3.17 (m, 10H), 1.21 (t, J=7.2 Hz, 3H).

### F. Synthesis of Compound 3

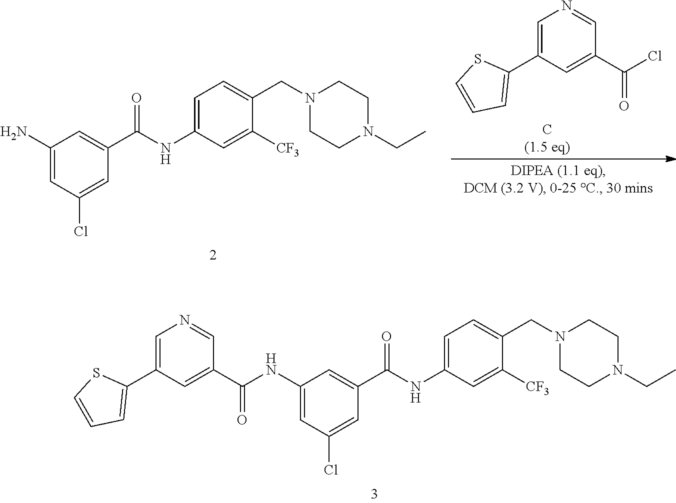 [SMILES: CCN1CCN(Cc2ccc(NC(=O)c3cc(N)cc(Cl)c3)cc2C(F)(F)F)CC1 ; O=C(Cl)c1cncc(-c2cccs2)c1 ; CCN1CCN(Cc2ccc(NC(=O)c3cc(Cl)cc(NC(=O)c4cncc(-c5cccs5)c4)c3)cc2C(F)(F)F)CC1]

To a solution of Compound 2 (3.20 g, 7.26 mmol, 1.0 eq) in DCM (10 mL) was added. Compound C (2.44 g, 10.9 mmol, 1.5 eq) and DIPEA (1.03 g, 7.98 mmol, 1.39 mL, 1.1 eq) at 0° C., and the mixture was stirred at 25° C. for 30 minutes. LC-MS showed that Compound 2 was consumed completely and one main peak with desired MS was detected. The reaction was poured into water (15 mL) and the reaction was extracted with ethyl acetate (50 mL, 30 mL). The reaction mixture was dried over Na₂SO₄, filtered and concentrated under reduced pressure to give a residue. The residue was purified by preparatory HPLC to give Compound 3 (1.6 g, 2.41 mmol, 33.2% yield) as a brown solid. ¹H NMR (400 MHZ, DMSO): δ (ppm): 10.87 (s, 1H), 10.64 (s, 1H), 9.12 (d, J=2.4, 1H), 9.03 (d, J=2.0 Hz, 1H), 8.53 (t, J=2.0 Hz, 1H), 8.27 (t, J=1.6 Hz, 1H), 8.22 (t, J=2.0 Hz, 1H), 8.18 (d, J=2.0 Hz, 1H), 8.04-8.07 (m, 1H) 7.86 (t, J=1.6 Hz, 1H), 7.79 (dd, J=3.6, 0.8 Hz, 1H), 7.72-7.75 (m, 2H), 7.24-7.27 (m, 1H), 3.57 (s, 2H), 2.29-2.40 (m, 10H), 0.98 (t, J=7.2 Hz, 3H).

### G. Synthesis of Compound TL5-135, Hydrochloride Salt

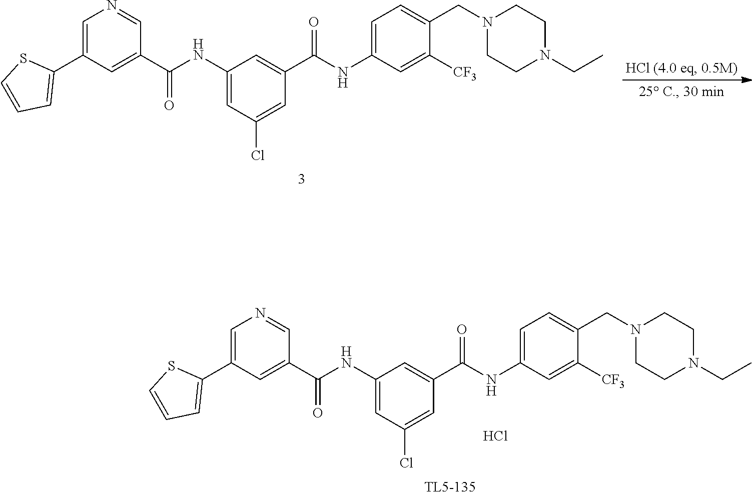 [SMILES: CCN1CCN(Cc2ccc(NC(=O)c3cc(Cl)cc(NC(=O)c4cncc(-c5cccs5)c4)c3)cc2C(F)(F)F)CC1 ; CCN1CCN(Cc2ccc(NC(=O)c3cc(Cl)cc(NC(=O)c4cncc(-c5cccs5)c4)c3)cc2C(F)(F)F)CC1 ; Cl]

A solution of Compound 3 (1.60 g, 2.55 mmol, 1.0 eq) in H₂O (5 mL) and HCl (0.5 M, 20.4 mL, 4.0 eq) was stirred at 25° C. for 30 minutes. The solution then was lyophilized to give TL5-135 (1.05 g, 1.42 mmol, 55.8% yield, HCl). ¹H NMR (400 MHZ, DMSO): δ (ppm): 11.88 (br, s, 1H), 11.27 (s, 1H), 10.93 (s, 1H), 9.18 (d, J=1.6 Hz, 1H), 9.13 (d, J=1.6 Hz, 1H), 8.82 (t, J=1.6 Hz, 1H), 8.39 (s, 1H), 8.35 (s, 1H), 8.29 (t, J=1.6 Hz, 1H), 8.20-8.22 (m, 2H), 7.90-7.92 (m, 2H), 7.76 (dd, J=5.2, 1.2 Hz, 1H), 7.25-7.27 (m, 1H), 4.40 (s, 2H), 3.18-3.68 (m, 10H), 1.26 (t, J=7.2 Hz, 3H).

### H. Synthesis of Compounds LSW-03-006, 007, 008, 015, and 072

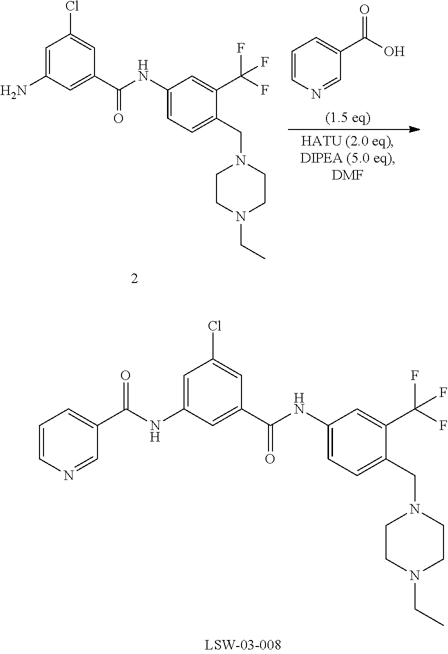

To a solution of Compound 2 (44 mg, 0.10 mmol, 1.0 eq) and nicotinic acid (18 mg, 0.15 mmol, 1.5 eq) in DMF (2 mL), 1-[Bis(dimethylamino)methylene]-1H-1,2,3-triazolo[4,5-b]pyridinium 3-oxide hexafluorophosphate (HATU) (76 mg, 0.20 mmol, 2.0 eq) and DIPEA (64 mg, 0.50 mmol, 5.0 eq) was added at 0° C. This mixture then was stirred at 25° C. for 12 hours. LC-MS showed that Compound 2 was consumed completely and one main peak with the desired MS was detected. The reaction was poured into water (15 mL) and was extracted with ethyl acetate (3×50 mL). The reaction mixture then was dried over Na₂SO₄, filtered and concentrated under reduced pressure to give a residue. The residue was purified by preparative HPLC to give Compound LSW-03-008 (28 mg, 0.051 mmol, 51.0% yield) as a brown solid. Compounds LSW-03-006, 007, 015, and 072 were synthesized using the same procedure. ¹H NMR (500 MHz, DMSO-d₆) δ 10.83 (s, 1H), 10.67 (s, 1H), 9.16 (d, J=2.3 Hz, 1H), 8.82 (dd, J=4.8, 1.7 Hz, 1H), 8.36 (dt, J=8.0, 2.0 Hz, 1H), 8.31 (t, J=1.8 Hz, 1H), 8.22-8.16 (m, 2H), 8.11 (dd, J=8.4, 2.2 Hz, 1H), 7.84 (t, J=1.8 Hz, 1H), 7.75 (d, J=8.5 Hz, 1H), 7.63 (dd, J=8.0, 4.9 Hz, 1H), 3.71 (s, 2H), 3.47 (d, J=12.0 Hz, 2H), 3.16 (q, J=7.4 Hz, 2H), 3.08-2.90 (m, 4H), 2.41 (d, J=12.2 Hz, 2H), 1.22 (t, J=7.3 Hz, 3H).

### I. Synthesis of Compound LSW-03-014

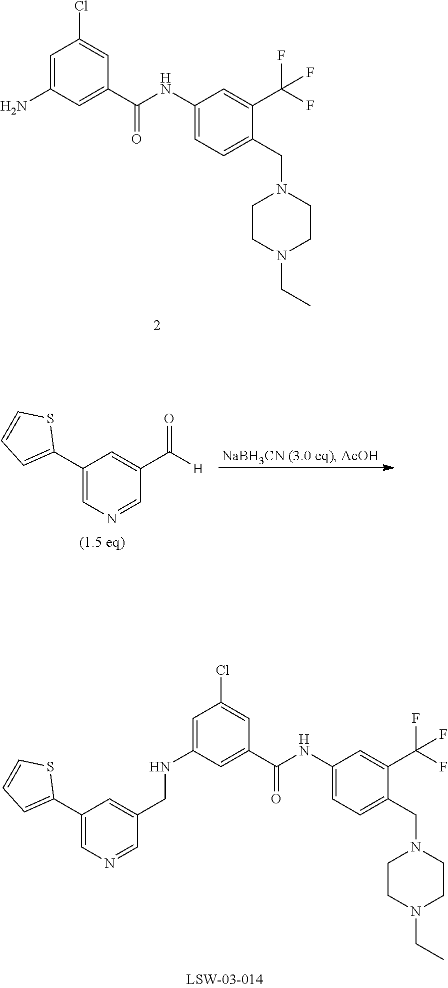 [SMILES: CCN1CCN(Cc2ccc(NC(=O)c3cc(N)cc(Cl)c3)cc2C(F)(F)F)CC1 ; O=Cc1cncc(-c2cccs2)c1 ; CCN1CCN(Cc2ccc(NC(=O)c3cc(Cl)cc(NCc4cncc(-c5cccs5)c4)c3)cc2C(F)(F)F)CC1]

To a solution of Compound 2 (22 mg, 0.05 mmol, 1.0 eq) and 5-(thiophen-2-yl) nicotinaldehyde (14 mg, 0.075 mmol, 1.5 eq) in AcOH (1 mL), NaBH₃CN (9.3 mg, 0.15 mmol, 3.0 eq) was added at 0° C. The mixture then was stirred at 25° C. for 2 hours. LC-MS showed that Compound 2 was consumed completely and one main peak with desired MS was detected. The reaction was poured into saturated aqueous sodium bicarbonate (20 mL) and the reaction was extracted with chloroform/isopropanol (v/v=4:1, 3×50 mL). The reaction mixture then was dried over Na₂SO₄, filtered and concentrated under reduced pressure to give a residue. The residue was purified by preparative HPLC to give Compound LSW-03-014 (15 mg, 0.024 mmol, 49.0% yield) as a white powder. ¹H NMR (500 MHZ, DMSO-d₆) δ 10.46 (s, 1H), 9.30 (s, 1H), 8.87 (d, J=2.2 Hz, 1H), 8.55 (d, J=1.9 Hz, 1H), 8.18 (d, J=2.2 Hz, 1H), 8.12 (t, J=2.2 Hz, 1H), 8.06 (dd, J=8.5, 2.2 Hz, 1H), 7.73-7.63 (m, 3H), 7.24-7.17 (m, 2H), 7.15 (t, J=1.9 Hz, 1H), 6.89 (t, J=2.0 Hz, 1H), 4.48 (s, 2H), 3.69 (s, 2H), 3.47 (d, J=12.0 Hz, 2H), 3.15 (q, J=7.3 Hz, 2H), 3.05-2.88 (m, 4H), 2.44-2.35 (m, 2H), 1.21 (t, J=7.3 Hz, 3H).

### J. Synthesis of Compound LSW-03-080

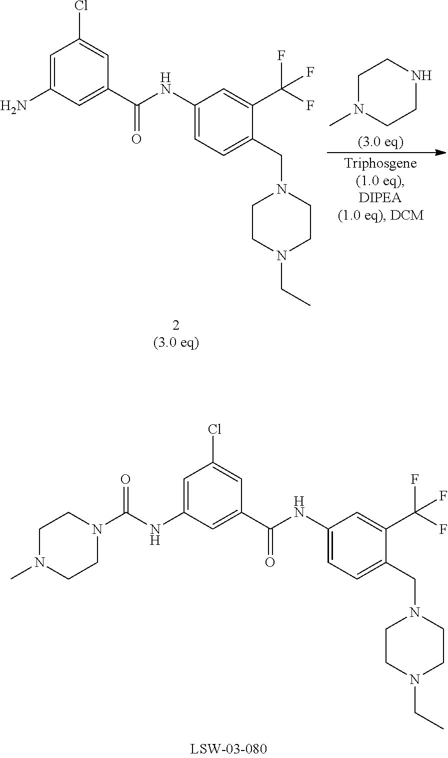

To a solution of Triphosgene (20 mg, 0.067 mmol, 1.0 eq) in DCM (2 mL), DIPEA (8.7 mg, 0.067 mmol, 1.0 eq) was added at 0° C., and the mixture then was stirred at 25° C. for 5 minutes. Compound 2 (89 mg, 0.20 mmol, 3.0 eq) was added to the reaction mixture and stirred at 25° C. for 30 minutes. 1-methylpiperazine (20 mg, 0.20 mmol, 3.0 eq) was added to the reaction mixture and stirred for 2 hours at 25° C. LC-MS showed that one main peak was detected with desired MS. The reaction was poured into water (15 mL) and the reaction was extracted with chloroform/isopropanol (v/v=4:1, 3×50 mL). The reaction mixture was dried over Na₂SO₄, filtered, and concentrated under reduced pressure to give a residue. The residue was purified by preparative HPLC to give Compound LSW-03-080 (12 mg, 0.021 mmol, 31.0% yield) as a white powder. MS (ESI): calcd. for C₂₇H₃₅ClF₃N₆O₂ [M+H]⁺567.25, found 567.33.

### K. Synthesis of Compound LSW-03-085

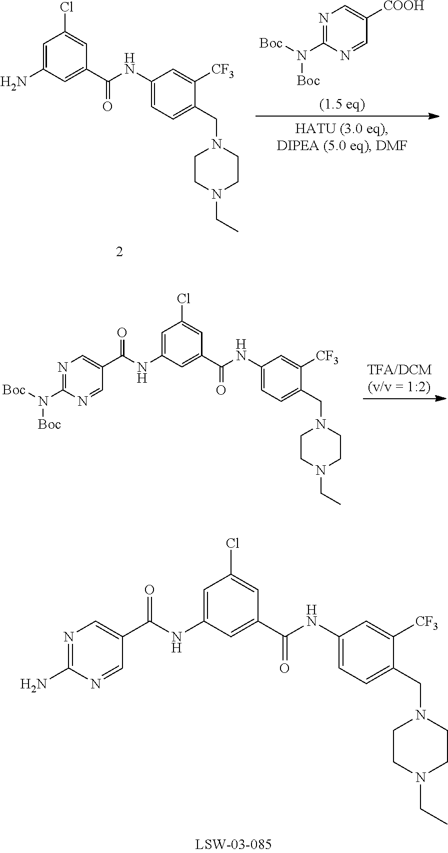 [SMILES: CCN1CCN(Cc2ccc(NC(=O)c3cc(N)cc(Cl)c3)cc2C(F)(F)F)CC1 ; CC(C)(C)OC(=O)N(C(=O)OC(C)(C)C)c1ncc(C(=O)O)cn1 ; CCN1CCN(Cc2ccc(NC(=O)c3cc(Cl)cc(NC(=O)c4cnc(N(C(=O)OC(C)(C)C)C(=O)OC(C)(C)C)nc4)c3)cc2C(F)(F)F)CC1 ; CCN1CCN(Cc2ccc(NC(=O)c3cc(Cl)cc(NC(=O)c4cnc(N)nc4)c3)cc2C(F)(F)F)CC1]

To a solution of Compound 2 (60 mg, 0.14 mmol, 1.0 eq) and 2-(bis(tert-butoxycarbonyl)amino)pyrimidine-5-carboxylic acid (69 mg, 0.20 mmol, 1.5 eq) in DMF (3 mL), 1-[Bis(dimethylamino)methylene]-1H-1,2,3-triazolo[4,5-b]pyridinium 3-oxide hexafluorophosphate (HATU) (160 mg, 0.41 mmol, 3.0 eq) and N,N-Diisopropylethylamine (DIPEA) (88 mg, 0.68 mmol, 5.0 eq) was added at 0° C. The mixture then was stirred at 25° C. for 12 hours. LC-MS showed that Compound 2 was consumed completely and one main peak with desired MS was detected. The reaction was poured into water (15 mL) and the reaction was extracted with ethyl acetate (3×50 mL). The reaction mixture was dried over Na₂SO₄, filtered and concentrated under reduced pressure to give a residue.

To a solution of the above residue in DCM (4 mL), TFA (2 mL) was added at 0° C. The mixture then was stirred at 25° C. for 1 hour. LC-MS showed that one main peak with desired MS was detected. The reaction solution was concentrated under reduced pressure to give a residue. The residue was purified by prep-HPLC to give Compound LSW-03-085 (28 mg, 0.037 mmol, 27.0% yield) as a white powder. ¹H NMR (500 MHz, DMSO-d₆) δ 10.65 (s, 1H), 10.39 (s, 1H), 8.84 (s, 2H), 8.25 (t, J=1.8 Hz, 1H), 8.20 (d, J=2.2 Hz, 1H), 8.16-8.08 (m, 2H), 7.79 (t, J=1.8 Hz, 1H), 7.74 (d, J=8.5 Hz, 1H), 7.44 (s, 2H), 3.70 (s, 2H), 3.47 (d, J=12.0 Hz, 2H), 3.16 (q, J=7.5 Hz, 2H), 2.98 (dd, J=25.0, 11.4 Hz, 4H), 2.41 (dd, J=12.2 Hz, 2H), 1.22 (t, J=7.3 Hz, 3H).

### L. MS and NMR Data

MS and NMR data for compounds is contained in Table 3, below.

TABLE 3
Selected MS and NMR Data.

| | LC/MS | | |
|---|---|---|---|
| | Retention | | |
| | Time | | MS m/z, |
| Compound | (min) | ¹H NMR chemical shift (ppm) | [M + 1]⁺ |
| LSW-03-006 | 1.02 | | 612.24 |
| LSW-03-007 | 0.83 | ¹H NMR (500 MHz, DMSO-d₆) δ 10.78 (s, 1H), 10.67 | 560.28 |
| | | (s, 1H), 8.97 (d, J = 2.1 Hz, 1H), 8.67 (d, J = 2.0 Hz, | |
| | | 1H), 8.31 (t, J = 1.8 Hz, 1H), 8.22-8.15 (m, 3H), 8.11 | |
| | | (dd, J = 8.5, 2.2 Hz, 1H), 7.84 (t, J = 1.8 Hz, 1H), 7.75 | |
| | | (d, J = 8.5 Hz, 1H), 3.70 (s, 2H), 3.47 (d, J = 12.0 Hz, | |
| | | 2H), 3.16 (q, J = 7.5, 6.6 Hz, 2H), 2.98 (dd, J = 26.5, | |
| | | 11.8 Hz, 4H), 2.43 (s, 3H), 2.41-2.35 (m, 2H), 1.22 (t, | |
| | | J = 7.3 Hz, 3H). | |
| LSW-03-008 | 0.77 | ¹H NMR (500 MHz, DMSO-d₆) δ 10.83 (s, 1H), 10.67 | 546.23 |
| | | (s, 1H), 9.16 (d, J = 2.3 Hz, 1H), 8.82 (dd, J = 4.8, 1.7 | |
| | | Hz, 1H), 8.36 (dt, J = 8.0, 2.0 Hz, 1H), 8.31 (t, J = 1.8 | |
| | | Hz, 1H), 8.22-8.16 (m, 2H), 8.11 (dd, J = 8.4, 2.2 Hz, | |
| | | 1H), 7.84 (t, J = 1.8 Hz, 1H), 7.75 (d, J = 8.5 Hz, 1H), | |
| | | 7.63 (dd, J = 8.0, 4.9 Hz, 1H), 3.71 (s, 2H), 3.47 (d, J = | |
| | | 12.0 Hz, 2H), 3.16 (q, J = 7.4 Hz, 2H), 3.08-2.90 (m, | |
| | | 4H), 2.41 (d, J = 12.2 Hz, 2H), 1.22 (t, J = 7.3 Hz, 3H). | |
| LSW-03-014 | 0.96 | ¹H NMR (500 MHz, DMSO-d₆) δ 10.46 (s, 1H), 9.30 | 614.24 |
| | | (s, 1H), 8.87 (d, J = 2.2 Hz, 1H), 8.55 (d, J = 1.9 Hz, | |
| | | 1H), 8.18 (d, J = 2.2 Hz, 1H), 8.12 (t, J = 2.2 Hz, 1H), | |
| | | 8.06 (dd, J = 8.5, 2.2 Hz, 1H), 7.73-7.63 (m, 3H), | |
| | | 7.24-7.17 (m, 2H), 7.15 (t, J = 1.9 Hz, 1H), 6.89 (t, | |
| | | J = 2.0 Hz, 1H), 4.48 (s, 2H), 3.69 (s, 2H), 3.47 (d, | |
| | | J = 12.0 Hz, 2H), 3.15 (q, J = 7.3 Hz, 2H), 3.05-2.88 | |
| | | (m, 4H), 2.44-2.35 (m, 2H), 1.21 (t, J = 7.3 Hz, 3H). | |
| LSW-03-015 | 1.15 | | 627.30 |
| LSW-03-030 | 0.84 | | 560.18 |
| LSW-03-033 | 1.55 | ¹H NMR (500 MHz, DMSO-d₆) δ 10.89 (s, 1H), 10.69 | 502.12 |
| | | (s, 1H), 9.13 (d, J = 2.2 Hz, 1H), 9.04 (d, J = 2.0 Hz, | |
| | | 1H), 8.54 (t, J = 2.2 Hz, 1H), 8.28 (t, J = 1.8 Hz, 1H), | |
| | | 8.23 (dt, J = 5.8, 1.9 Hz, 2H), 8.08 (dd, J = 8.1, 2.0 Hz, | |
| | | 1H), 7.87 (t, J = 1.8 Hz, 1H), 7.80 (dd, J = 3.7, 1.2 Hz, | |
| | | 1H), 7.75 (dd, J = 5.1, 1.1 Hz, 1H), 7.64 (t, J = 8.0 Hz, | |
| | | 1H), 7.50 (d, J = 7.8 Hz, 1H), 7.27 (dd, J = 5.0, 3.6 Hz, | |
| | | 1H). | |
| LSW-03-054 | 1.03 | | 612.24 |
| LSW-05-055 | 1.12 | | 662.35 |
| LSW-05-059 | 1.05 | | 646.35 |
| LSW-05-067 | 0.93 | | 612.24 |
| LSW-03-071 | 1.09 | | 674.21 |
| LSW-03-072 | 1.06 | ¹H NMR (500 MHz, DMSO-d₆) δ 10.96 (s, 1H), 10.65 | 547.23 |
| | | (s, 1H), 9.41 (s, 1H), 9.31 (s, 2H), 8.24 (t, J = 1.8 Hz, | |
| | | 1H), 8.21-8.16 (m, 2H), 8.06 (dd, J = 8.3, 2.3 Hz, | |
| | | 1H), 7.88 (t, J = 1.7 Hz, 1H), 7.74 (d, J = 8.5 Hz, 1H), | |
| | | 3.59 (s, 2H), 2.46-2.23 (m, 10H), 1.00 (t, J = 7.2 Hz, | |
| | | 3H). | |
| LSW-03-077 | 1.22 | ¹H NMR (500 MHz, DMSO-d₆) δ 10.88 (s, 1H), 10.68 | 647.40 |
| | | (s, 1H), 9.21 (d, J = 2.3 Hz, 1H), 9.19 (s, 1H), 8.70 (t, | |
| | | J = 2.1 Hz, 1H), 8.31 (t, J = 1.7 Hz, 1H), 8.23-8.18 (m, | |
| | | 2H), 8.15-8.03 (m, 5H), 7.87 (t, J = 1.7 Hz, 1H), 7.75 | |
| | | (d, J = 8.5 Hz, 1H), 3.70 (s, 2H), 3.47 (d, J = 12.0 Hz, | |
| | | 2H), 3.19-3.13 (m, 2H), 2.97 (dd, J = 28.4, 12.4 Hz, | |
| | | 4H), 2.40 (d, J = 12.1 Hz, 2H), 1.22 (t, J = 7.3 Hz, 3H). | |
| LSW-03-080 | 0.80 | | 567.33 |
| LSW-03-081 | 1.13 | | 520.27 |
| LSW-03-082 | 1.31 | | 622.25 |
| LSW-03-083 | 1.16 | | 571.24 |
| LSW-03-085 | 1.05 | ¹H NMR (500 MHz, DMSO-d₆) δ 10.65 (s, 1H), 10.39 | 562.23 |
| | | (s, 1H), 8.84 (s, 2H), 8.25 (t, J = 1.8 Hz, 1H), 8.20 (d, | |
| | | J = 2.2 Hz, 1H), 8.16-8.08 (m, 2H), 7.79 (t, J = 1.8 Hz, | |
| | | 1H), 7.74 (d, J = 8.5 Hz, 1H), 7.44 (s, 2H), 3.70 (s, | |
| | | 2H), 3.47 (d, J = 12.0 Hz, 2H), 3.16 (q, J = 7.5 Hz, | |
| | | 2H), 2.98 (dd, J = 25.0, 11.4 Hz, 4H), 2.41 (dd, J = | |
| | | 12.2 Hz, 2H), 1.22 (t, J = 7.3 Hz, 3H). | |
| LSW-03-086 | | | |

### Example 3. Screening of the Nathanael Gray Diverse Kinase Inhibitor Libraries

To establish the utility of kinase inhibitor compounds synthesized in the Gray laboratory as a source of novel antimalarial activities, the libraries (containing 1,100 compounds) were screened using the well-established SYBR Green I-based fluorescence assay as known in the art. This screen yielded 34 compounds with EC₅₀<500 nM in the CQ^{R} Dd2 strain. These hits were subjected to counter-screening against human HepG2 cells using CellTiter 96® AQueous (Promega™) MTS-based cell proliferation assay to identify asexual and liver stage antiplasmodial hits.

After down-selecting compounds based on novelty, potency, and selectivity, the compound TL5-135 was identified as the most potent hit compound from the screen. See Table 4, below. P. falciparum Dd2 EC₅₀ was determined using SYBR Green I assay. For the selectivity index (Dd2 EC₅₀/HepG2 human liver cell EC₅₀) cytotoxicity was determined using CellTiter 96® AQueous MTS. The liver stage assay was performed using P. berghei luciferase expressing parasites. All assays were performed with technical quadruplicates and 3 biological replicates. Assays have a Z′ factor >0.7.

TABLE 4
TL5-135 Screening Results (Antiplasmodial Hits).

| | PfDd2 EC₅₀ | Selectivity | Liver Stage |
|---|---|---|---|
| Compound | (nM) ± SD | Index (SI) | EC₅₀ (nM) ± SD |
| TL5-135 | 80 ± 8 | 116 | 108 ± 35 |

### Example 4. TL5-135 Exhibits Therapeutic and Prophylactic Activities

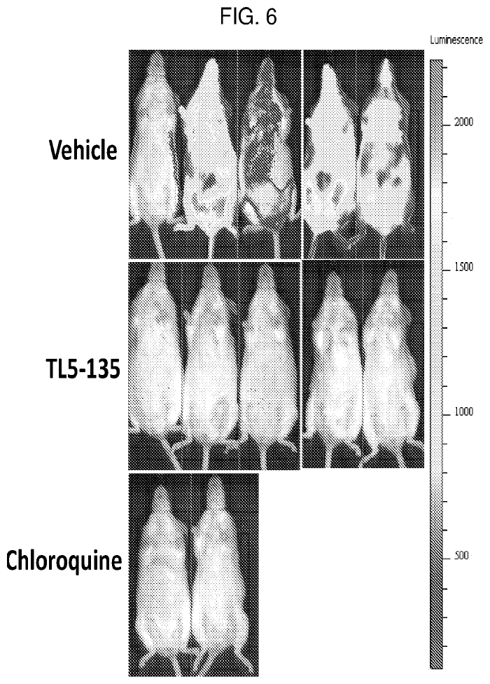

Mice infected with P. berghei ANKA (10³ iRBC) expressing luciferase were treated with 50 mg/kg TL5-135 or chloroquine orally once daily 72 hours post-infection for 4 days. Imaging was done 7 days after infection following a 150 mg/kg D-luciferin injection with an IVIS. As shown in FIG. 6, TL5-135 eliminates established P. berghei ANKA infection at 50 mg/kg p.o., administered orally once daily for 4 days.

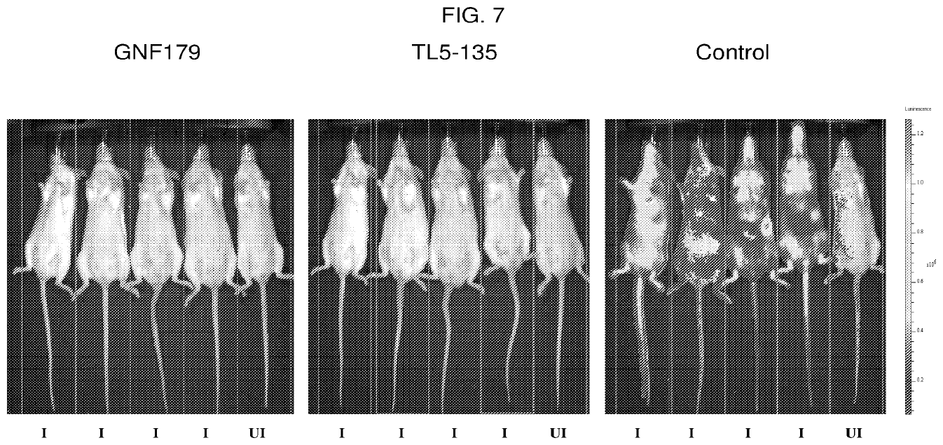

Further study showed that TL5-135 has prophylactic activity when administered as a single dose (50 mg/kg, IV) 6 hours prior to infection of mice with sporozoites. See FIG. 7. Infection was initiated using 100,000 P. berghei luc sporozoites (Pb-Luc). The drugs were administered IV at 50 mg/kg, single dose 6 hours prior. I: infected, UI: uninfected.

These results establish the utility of TL5-135 as a lead compound from which different derivatives or analogs can be synthesized to find prophylactic and therapeutic compounds.

### Example 5. Prophylactic and Therapeutic Activity

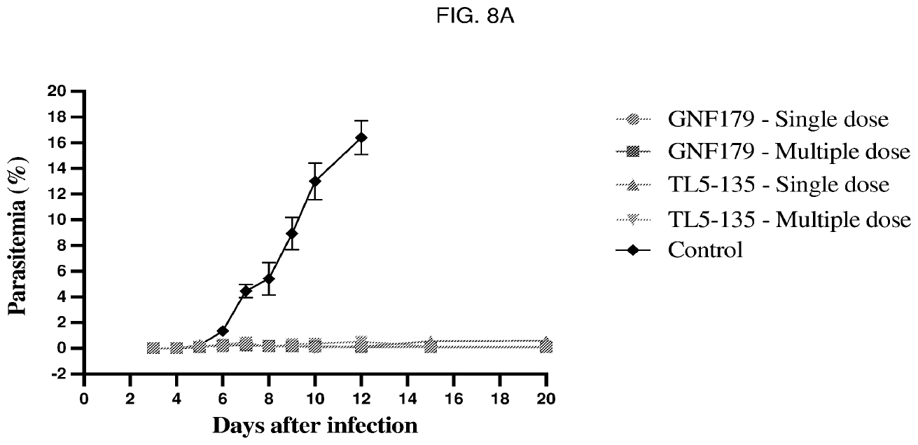

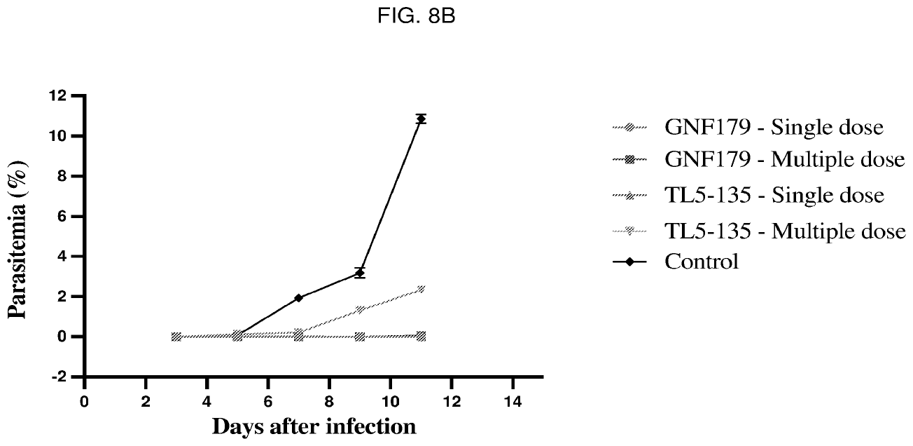

FIG. 8A shows the results of tests comparing TL5-135 and GNF179 (positive control) antimalarial compounds against Pb-Luc sporozoites. Groups of 5 mice were infected with 100,000 Pb-Luc sporozoites by IV injection. Prophylactic (single dose of 50 mg/kg through IV injection, 6 hour before infection) and therapeutic (4-day Peters test, 4 doses of 15 mg/kg each time were administrated post-infection at 6, 18, 30, and 42 hours post infection (hpi) and the activity of the compounds was evaluated. See FIG. 8A. The parasitemia was estimated after flow cytometric enumeration of rodent malaria parasite Pb-Luc-infected red blood cells (RBS) stained with SYBR Green I, from day 3 until 20.

FIG. 8B shows the results of a second test of the prophylactic and therapeutic efficacy of TL5-135 at a lower dose of 15 mg/kg. TL5-135 and GNF179 antimalarial compounds were tested against Pb-Luc sporozoites. Prophylactic activity (single dose of 15 mg/kg through an IV injection, 6 hours before infection) and therapeutic activity (4-day Peters test, 4 doses of 15 mg/kg each time was administrated at 6, 18, 30, and 42 hpi of the TL5-135 antimalarial compound was evaluated. As before, groups of five mice were infected with 100,000 Pb-Luc sporozoites by IV injection. The parasitemia was estimated after flow cytometric enumeration of rodent malaria parasite Pb-Luc-infected red blood cells (RBS) stained with SYBR Green I, from day 3 until day 20. See FIG. 8B.

### Example 6. Mouse Survival Curves

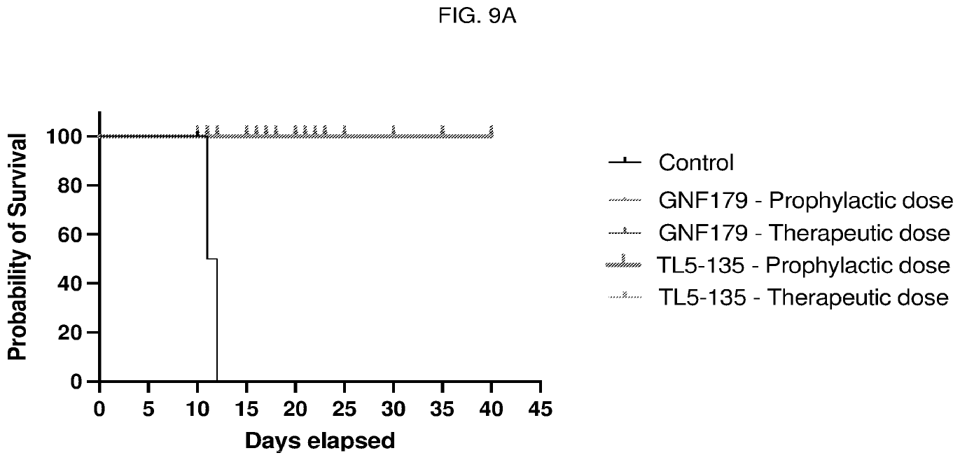

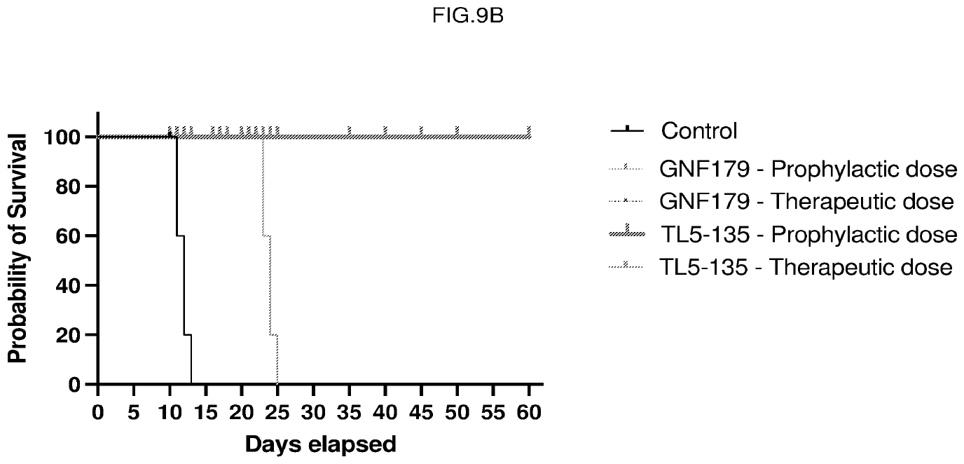

A study was performed to compare infected mouse survival when treated with TK59135. For treatment, TL5-135 was dissolved in a vehicle containing 27% DMSO, 1% Methylcellulose, 0.5% Tween-80 and 71.5% ultrapure water; GNF179 was resuspended in 34% DMSO, 25% PEG400, 5% Tween-80, 36% saline, and administered intravenously. Dosing of the treatment compounds was the same as above, i.e., 50 mg/kg for the results in FIG. 9A and 15 mg/kg for the results in FIG. 9B.

FIG. 9A is a survival curve of mice (Swiss webster) infected with Pb-Luc parasites for 90 days after infection, showing the results. The study compared infected mouse survival among the untreated (negative control) and treated groups (GNF179 positive control and TL5-135) at a prophylactic single dose and therapeutic multiple doses (p<0.0001, Log-rank (Mantel-Cox) test (n=5 at each group treated)). The results are expressed as the mean±SEM.

FIG. 9B is a second survival curve of mice (Swiss webster) infected with Pb-Luc parasites for 90 days after infection. Comparison of infected mouse survival between the untreated (Negative control) and treated groups (GNF179-prophylactic single dose, GNF179-Therapeutic multiple doses, TL5-135-Prophylactic single dose, and TL5-135-Therapeutic multiple doses) is presented (p<0.0001, Log-rank (Mantel-Cox) test (n=5 at each group treated)). The results are expressed as the mean±SEM.

At 50 mg/kg (FIG. 9A), infected animals survived for 40 days with no signs of recrudescence of parasites.

### Example 7. Determination of the Stage-Specific Action of Compounds

The stage specific action of the prioritized hits was determined as follows. Synchronized cultures of P. falciparum strain Dd2 were exposed to 400 nM (5×EC₅₀) compound at 6 hours post infection for 12 hours and fixed samples labeled with YOYO-1 dye were analyzed by flow cytometry and microscopy using Giemsa-stained thin smears.

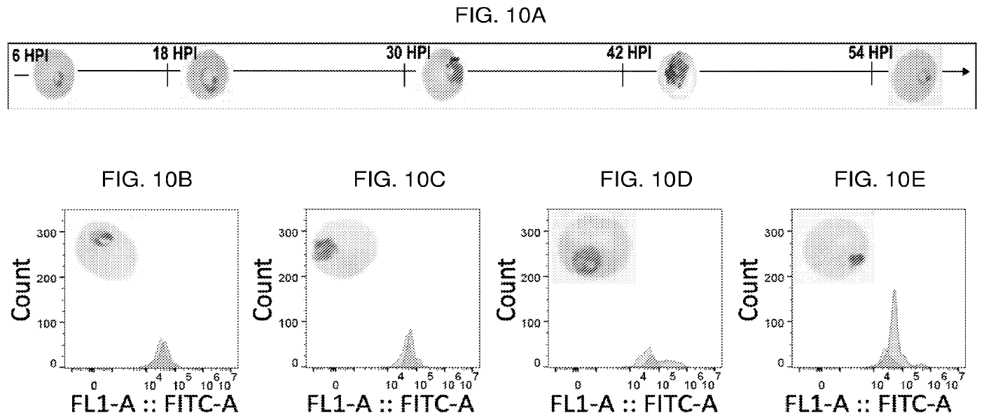

As shown in FIG. 10, the compound TL5-135 blocks ring to trophozoite maturation when treated at early ring stage. This is significant because. other than artemisinin, none of the current antimalarials act early at the ring stage. TL5-135 also blocks maturation of asexual parasites when added at the trophozoite and schizont stages (data not shown). FIG. 10B, FIG. 10C, FIG. 10D and FIG. 10E show microscopic images and merged flow cytometric profiles for TL5-135-treated samples. The results are representative of 3 biological replicates.

The results shown in FIG. 9 and FIG. 10 demonstrate that TL5-135 has both prophylactic and therapeutic activities. TL5-135 inhibits asexual stage maturation early, exhibits a fast killing rate, and exhibits metabolic stability in the subject, making it a useful compound in the treatment and prophylaxis of malarial infection.

### Example 8. Parasite Rate of Killing Assay Results

Asynchronous cultures of P. falciparum Dd2 parasites were treated with 10×EC₅₀ of the indicated compound for 12 hours prior to compound wash off. Flow cytometry with SYBR Green I (nuclear dye) and Mitotracker Deep Red FM (mitochondria staining) was done after compound wash-off every 24 hours after for 6 days (100,000 events/sample) to track parasitemia (data from 3 biological replicates±SEM).

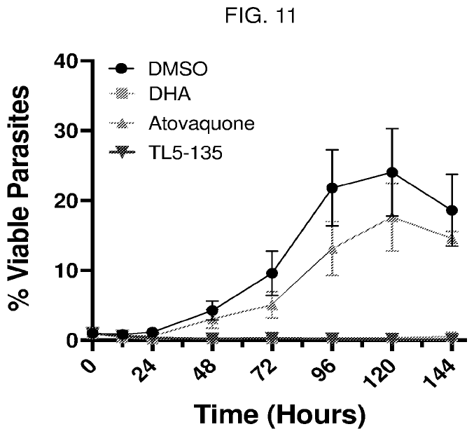

As shown in FIG. 11, TL5-135 exhibited pronounced irreversible killing following a 12-hour exposure as seen with the dihydroartemisinin (DHA) positive control. TL5-135 also exhibited desirable microsomal stability in mouse liver microsomes (t_{1/2}=79.5 min).

In summary, TL5-135 meets many of the MMV (Medicine for Malaria Venture) Target Product Profiles (TPP) criteria as it shows activity against multiple stages pf parasite development with mechanism of action different from current antimalarials, acts rapidly, and with acceptable PK criteria for parasite clearance.

1. A compound of Formula I: 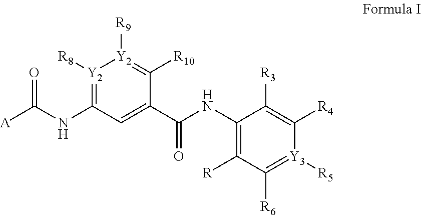
or a prodrug, stereoisomer, or pharmaceutically acceptable salt thereof,
wherein A is 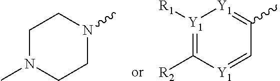
wherein each Y₁ is C or optionally one or two of Y₁ is N, wherein R¹ is no group, —H, —CH₃, —CN, 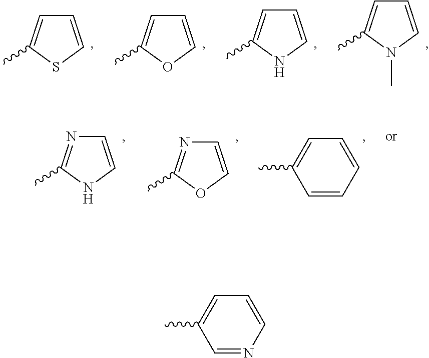
and wherein R₂ is no group, —H, or —NH₂;
wherein Y₂ is C or optionally one of Y₂ is N;
wherein Y₃ is C or N;
wherein R³ is —H;
wherein R⁴ is —H, —CF₃, 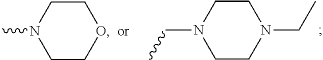
wherein R⁵ is no group, —H, or 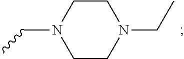
wherein R⁶ is —H, —CF₃, 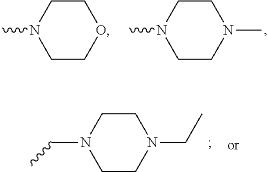 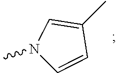
wherein R₇ is —H or —X;
wherein R₈ is no group, —H, —CH₃, or —X;
wherein R₉ is no group, —H, —CH₃, —C₂H₅, —CF₃, —CN, —OCH₃, or —X;
wherein R₁₀ is —H or —X; and
wherein X is-F, —Cl, or —Br.

2. The compound of claim 1 according to Formula IIA or Formula IIB 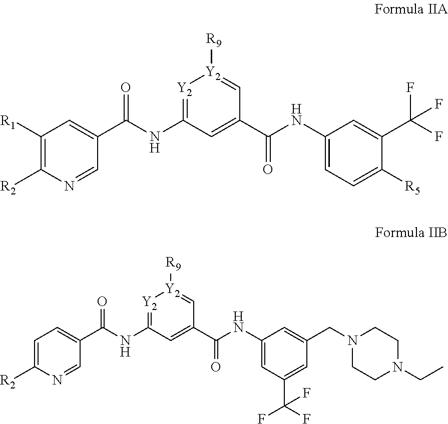
wherein R¹ is —H, —CN, 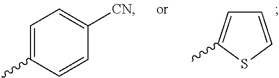
wherein R₂ is —H or —NH₂;
wherein Y₂ is C or optionally one of Y₂ is N;
wherein R⁵ is —H or 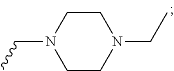
and
wherein R₉ is —H, —X, CH₃, or —OCH₃, or is absent when one of Y₂ is N.

3. The compound of claim 1 according to Formula III 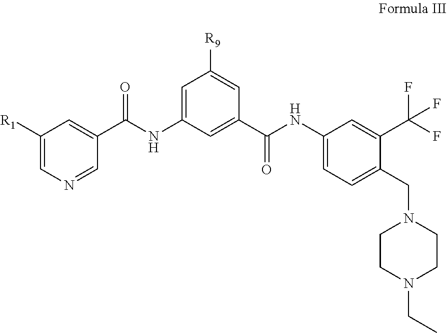
wherein R₉ is —H, —CH₃, —C₂H₅, —CF₃ or —X, wherein X is —Cl, —F, or —Br; and
wherein R¹ is —H, —CN,

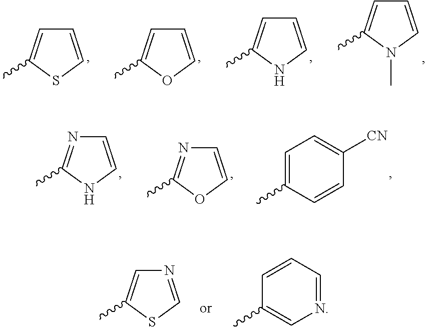

4. The compound of claim 1 according to Formula IV 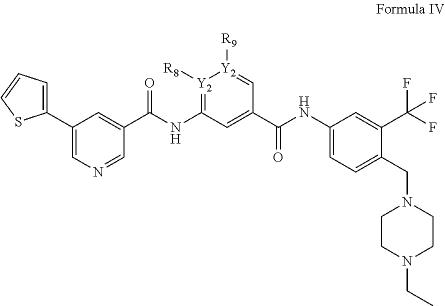
wherein Y₂ is C or optionally one of Y₂ is N;
wherein R₈ is —H or —CH₃; and
wherein R₉ is —CH₃, —C₂H₅, or —CN.

5. The compound of claim 1 according to Formula V 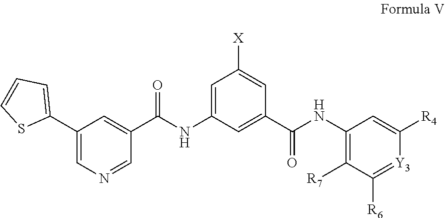
wherein Y₃ is C or N;
wherein R⁴ is-CF₃ or 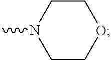
wherein R⁶ is —H, 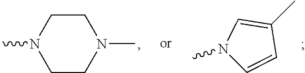
and
wherein R₇ is —H or —X, wherein X is —Cl, —F, or —Br.

6. A compound according to Formula VI 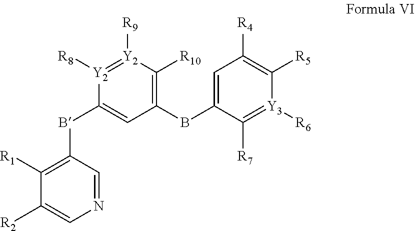
wherein R¹ is no group, —H, —CH₃, —CN, 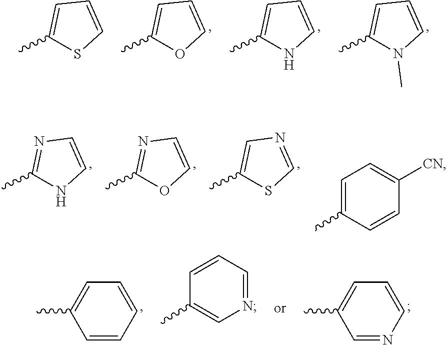
wherein R₂ is —H or —NH;
wherein B is —CO—NH—, —NH—CO—, or —NH—;
wherein B′ is —CO—NH—, —NH—CO—, or —NH—;
wherein Y₂ is C or optionally one of Y₂ is N;
wherein Y₃ is C or N;
wherein R⁴ is —H, —CF₃, 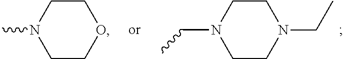
wherein R⁵ is —H, —CH₃, or 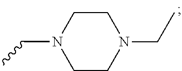
wherein R⁶ is —H, —CF₃, 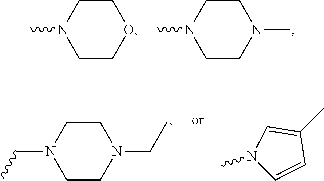
wherein R₇ is —H, —X;
wherein R₈ is no group, —H, —CH₃, or —X;
wherein R₉ is no group, —H, —CH₃, —C₂H₅, —CF₃, —CN, —OCH₃, or —X; and
wherein R₁₀ is —H or —X, wherein X is Cl, F, or Br.

7. The compound of claim 1 wherein A is 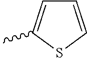
and R₅ is

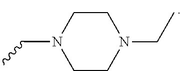

8. The compound of claim 1 which is

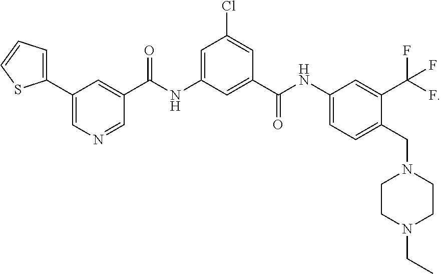 [SMILES: CCN1CCN(Cc2ccc(NC(=O)c3cc(Cl)cc(NC(=O)c4cncc(-c5cccs5)c4)c3)cc2C(F)(F)F)CC1]

9. A pharmaceutical composition comprising a pharmaceutically acceptable carrier and the compound of claim 1.

10. A pharmaceutical composition comprising a pharmaceutically acceptable carrier and the compound of claim 8.

12. The pharmaceutical composition of claim 9 which is formulated for oral administration.

13. The pharmaceutical composition of claim 9 which is formulated for administration by injection.

14. A method for treatment of prevention of Plasmodium infection in a subject in need thereof, comprising administering an effective amount of the compound of claim 9 to the subject in need.

15. The method of claim 14, wherein the Plasmodium comprises P. berghei, P. falciparum, P. vivax, P. ovale, P. malariae, or P. knowlesi.

16. A method of synthesizing a compound of any of claims 1-8.

17. The method of claim 16, wherein the method is as is disclosed in Example 2.
